(19) United States
(12) Patent Application Publication
(10) Pub. No.: US 2020/0121767 A1
(43) Pub. Date: Apr. 23, 2020

(54) METHODS FOR IDENTIFYING THE HEALTH STATE OF HYPOPHOSPHATASIA (HPP) PATIENTS

(71) Applicant: Alexion Pharmaceuticals, Inc., Boston, MA (US)

(72) Inventors: Ioannis C. TOMAZOS, Boston, MA (US); Andrew LLOYD, Boston, MA (US)

(73) Assignee: Alexion Pharmaceuticals, Inc., Boston, MA (US)

(21) Appl. No.: 16/603,927

(22) PCT Filed: Apr. 10, 2018
(86) PCT No.: PCT/US2018/026868

§ 371 Date: Oct. 9, 2019

Related U.S. Application Data
(60) Provisional application No. 62/555,384, filed on Sep. 7, 2017.
(60) Provisional application No. 62/485,214, filed on Apr. 13, 2017.

(51) Int. Cl. A61K 38/46; A61K 9/00; A61P 19/08; A61P 3/00; A61B 5/11; A61B 5/00; A61B 5/08; G16H 50/30

(52) U.S. Cl. CPC A61K 38/465; C12Y 301/03001; A61K 9/0019; A61P 19/08; A61P 3/00; G16H 50/30; A61B 5/1124; A61B 5/4824; A61B 5/4094; A61B 5/4504; A61B 5/0816; A61B 5/112

(57) ABSTRACT

The disclosure features methods for identifying the health state of patients having hypophosphatasia (HPP). The health state of the patient, once identified, can be used to, e.g., assign a treatment regimen featuring the administration of a soluble alkaline phosphatase (sALP) to the patient, to monitor the efficacy of the treatment regimen, and/or to modify the treatment regimen. The methods also include assessing the transition of a patient with HPP from one health state to another, e.g., after completion of a treatment regimen that includes administering an sALP.

### BACKGROUND

Hypophosphatasia (HPP) is a rare, heritable skeletal disease with an incidence of 1 per 100,000 births for the most severe forms of the disease. The disorder typically results from loss-of-function mutations in the gene coding for tissue-nonspecific alkaline phosphatase (TNALP). HPP exhibits a remarkable range of symptoms and severity, from premature tooth loss to almost complete absence of bone mineralization in utero. The presentation of HPP varies markedly among patients, and also varies markedly between patient ages. To date, a number of publications have focused on the more easily quantified increase in X-ray visible bones of infants affected with HPP, but quantitative quality of life (QoL) analyses have been lacking. Many patients having HPP display skeletal changes, short stature, chronic pain, painful lower limbs, gait disturbance, and premature, atraumatic tooth loss. Asfotase alfa (STRENSIQ®, Alexion Pharmaceuticals, Inc.), a tissue non-specific alkaline phosphatase fusion protein produced by recombinant DNA technology, is the first, and only, treatment available to patients diagnosed with HPP.

There exists a need for methods to characterize the health status of HPP patients prior to and during treatment so that the efficacy of the treatment regimen can be monitored and personalized, if desirable.

### SUMMARY

Disclosed are (1) methods to assign one of four health states to a patient with hypophosphatasia (HPP), (2) methods to identify a health state of a patient with HPP (e.g., children having HPP of about 5 years of age to about 12 years of age, adolescents having HPP of about 13 years of age to about 17 years of age, or adults having HPP of greater than about 18 years of age or older), and (3) methods to assign a treatment regimen based on a health state of a patient, such as treatment with a soluble alkaline phosphatase (sALP; such as TNALP, for example the sALP polypeptide of SEQ ID NO: 1 or a polypeptide variant having at least 95% sequence identity to the sequence of SEQ ID NO: 1, e.g., asfotase alfa).

A first aspect features a method of assigning one of four health states (e.g., the health state is I, II, III, or IV) to a patient with HPP that includes: (a) characterizing the physiological condition of the patient using one or more metrics; and (b) using results of the one or more metrics to assign the patient into one of the four health states.

A second aspect features a method of assigning a treatment regimen based on a health state (e.g., the health state is I, II, III, or IV) of a patient with HPP (e.g., a child having HPP of about 5 years of age to about 12 years of age, an adolescent having HPP of about 13 years of age to about 17 years of age, or an adult having HPP of greater than about 18 years of age or older) that includes: (a) characterizing the physiological condition of the patient using one or more metrics; (b) using results of the one or more metrics to identify the health state of the patient; and (c) assigning the treatment regimen based on the health state of the patient. In particular, the treatment regimen includes administering at least 0.5 mg/kg/week of a sALP (e.g., about 1 mg/kg/week to about 9 mg/kg/week, preferably 6 mg/kg/week, of the sALP) to the patient for a treatment period of at least two weeks (e.g., at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six months). The sALP includes an amino acid sequence having at least 95% sequence identity to the amino acid sequence of SEQ ID NO: 1 (e.g., the sALP includes or consists of SEQ ID NO: 1). In particular, the treatment period is at least 96 weeks.

A third aspect features a method of assessing transition of a patient with HPP from one health state (e.g., the health state is I, II, III, or IV) to another including: (a) characterizing the physiological condition of the patient using one or more metrics; (b) using results of the one or more metrics to identify the health state of the patient; and (c) assessing a change in the health state of the patient by repeating steps (a) and (b) one or more times after completion of a treatment regimen. In particular, the treatment regimen includes administration at least 0.5 mg/kg/week of a sALP (e.g., about 1 mg/kg/week to about 9 mg/kg/week, preferably 6 mg/kg/week, of the sALP) to the patient for a treatment period of at least two weeks (e.g., at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six months). The sALP includes an amino acid sequence having at least 95% sequence identity to the amino acid sequence of SEQ ID NO: 1 (e.g., the sALP includes or consists of SEQ ID NO: 1). In particular, the treatment period is at least 96 weeks.

In any of the above aspects, the treatment regimen improves the health state of the patient, particularly from IV to III, II, or I; or from III to II or I; or from II to I and/or the treatment regimen maintains the health state of the patient. The health state (e.g., a health state of I, II, III, or IV) of the patient with HPP (e.g., a child having HPP of about 5 years of age to about 12 years of age, an adolescent having HPP of about 13 years of age to about 17 years of age, or an adult having HPP of greater than about 18 years of age or older) can be characterized with at least one physical assessment selected from one or more of the following metrics: Six Minute Walk Test (6MWT), Bruininks-Oseretsky Test of Motor Proficiency 2nd Edition (BOT-2), Bayley Scales of Infant and Toddler Development, 3rd Edition (BSID-III), and gait analysis, and at least one quality of life assessment selected from one or more of the following metrics: EuroQol Five Dimension Questionnaire (EQ-5D), Childhood Health Assessment Questionnaire (CHAQ), Pediatric Outcomes Data Collection Instrument (PODCI), Child Health Utility Index-9D (CHU-9D), Pediatric Quality of Life Inventory (PedsQL), Short Form Health Survey 36 (SF-36), and Short Form Health Survey 12 (SF-12). In particular, the patient has an improvement in the health state to at least health state III, II, or I after administration of the sALP.

For example, the patient is about 5 years of age to about 12 years of age and the health state of the patient is characterized by performing at least one physical assessment selected from one or more of the following: 6MWT, BOT-2, gait analysis, and BSID-III, and at least on quality of life assessment selected from one or more of the following: EQ-5D, CHAQ, PODCI, CHU-9D, and PedsQL. In particular, prior to administration of the sALP: (i) the patient is identified as having health state IV when the patient has a 6 MWT value of less than about 47.2% of a predicted 6MWT value for a healthy subject; (ii) the patient is identified as having health state III when the patient has a 6MWT value of about 47.2% to about 64.8% of a predicted 6MWT value for a healthy subject; (iii) the patient is identified as having health state II when the patient has a 6MWT value of about 64.8% to about 82.4% of a predicted 6MWT value for a healthy subject; or (iv) the patient is identified as having health state I when the patient has a 6MWT value of greater than 82.4% of a predicted 6MWT value for a healthy subject. Moreover, after administration of the sALP, the patient has an improved health state. The method may further include assessing one or more of the following symptoms: elevated blood or urine levels of PPi, PEA, or PLP; rickets, rachitic ribs, one or more skeletal deformities, hypotonia, muscle weakness, rheumatoid complications, arthritis, pseudogout, waddling gait, ambulatory difficulties, bone pain, pain, premature loss of teeth, hypomineralization, delayed motor development, seizures, hypercalciuria, short stature, bone fracture, pseudofracture, and growth delay. In an embodiment, the health state of the patient is characterized by performing the 6MWT as the physical assessment and the EQ-5D as the quality of life assessment.

For instance, the patient is about 13 years of age to about 17 years of age and the health state of the patient is characterized by performing at least one physical assessment selected from one or more of the following: 6MWT, BOT-2, and gait analysis, and at least one quality of life assessment selected from one or more of the following: EQ-5D, CHAQ, and PODCI. In particular, prior to administration of the sALP: (i) the patient is identified as having health state IV when the patient has a 6MWT value of less than about 47.8% of a predicted 6MWT value for a healthy subject; (ii) the patient is identified as having health state III when the patient has a 6MWT value of about 47.8% to about 65.2% of a predicted 6MWT value for a healthy subject; (iii) the patient is identified as having health state II when the patient has a 6MWT value of about 65.2% to about 82.6% of a predicted 6MWT value for a healthy subject; or (iv) the patient is identified as having health state I when the patient has a 6MWT value of greater than 82.6% of a predicted 6MWT value for a healthy subject. Moreover, after administration of the sALP, the patient has an improved health state. The method may further include assessing one or more of the following symptoms: elevated blood or urine levels of PPi, PEA, or PLP; osteomalacia, one or more skeletal deformities, hypotonia, muscle weakness, rheumatoid complications, arthritis, pseudogout, waddling gait, ambulatory difficulties, bone pain, pain, premature loss of teeth, hypomineralization, pulmonary hypoplasia, respiratory insufficiency, seizures, hypercalciuria, short stature, and growth delay. In an embodiment, the health state of the patient is characterized by performing the 6MWT as the physical assessment and the EQ-5D as the quality of life assessment.

For example, the patient is about 18 years of age or older and the health state of the patient is characterized by performing at least one physical assessment selected from one or more of the following: the 6MWT, BOT-2, and gait analysis, and at least one quality of life assessment selected from one or more of the following: EQ-5D, SF-36, and SF-12. In particular, prior to administration of the sALP: (i) the patient is identified as having health state IV when the patient has a 6MWT value of less than about 52.0% of a predicted 6MWT value for a healthy subject; (ii) the patient is identified as having health state III when the patient has a 6MWT value of about 52.0% to about 68.0% of a predicted 6MWT value for a healthy subject; (iii) the patient is identified as having health state II when the patient has a 6MWT value of about 68.0% to about 84.0% of a predicted 6MWT value for a healthy subject; or (iv) the patient is identified as having health state I when the patient has a 6MWT value of greater than 84.0% of a predicted 6MWT value for a healthy subject. Moreover, after administration of the sALP, the patient has an improved health state. The method may further include assessing one or more of the following symptoms: elevated blood or urine levels of PPi, PEA, or PLP, hypomineralization, hypercalciuria, skeletal deformity, waddling gait, bone pain, bone fracture, calcium pyrophosphate dihydrate crystal deposition, arthritis, pyrophosphate arthropathy, chondrocalcinosis, calcific periarthritis, and pseudofracture. In an embodiment, the health state of the patient is characterized by performing the 6MWT as the physical assessment and the EQ-5D as the quality of life assessment.

In any of the above aspects, prior to administration of the sALP: (i) the patient is identified as having health state IV and the patient has an EQ-5D value of less than about 0.23; (ii) the patient is identified as having health state III and the patient has an EQ-5D value of about 0.23 to about 0.54; (iii) the patient is identified as having health state II and the patient has an EQ-5D value of about 0.54 to about 0.67; or (iv) the patient is identified as having health state I and the patient has an EQ-5D value of greater than about 0.67. Moreover, after administration of the sALP, the patient has an improved health state.

The method can further include administering the sALP (e.g., the sALP polypeptide of SEQ ID NO: 1 or a polypeptide variant having at least 95% sequence identity to the sequence of SEQ ID NO: 1, e.g., asfotase alfa) to the patient in a treatment regimen providing about 1 mg/kg/week to about 9 mg/kg/week, preferably 6 mg/kg/week. In particular, the patient exhibits an improvement in the health state to at least health state III, II, or I after administration of the sALP. For example, the sALP is administered at an initial dosage of about 2.1 mg/kg/week to about 3.5 mg/kg/week and subsequently is increased to a dosage of about 6 mg/kg/week to about 9 mg/kg/week in the treatment regimen. The sALP (e.g., SEQ ID NO: 1) may be administered one or more times per day, week, month, or year (e.g., twice a week, three times a week, four times a week, five times a week, six times a week, or seven times a week). In particular, the sALP may be administered in multiple doses on two days a week, three days a week, four days a week, five days a week, six days a week, or seven days a week. In particular, the initial dosage may be increased after a treatment period of at least six months, at least one year, at least two years, at least three years, or at least four years or longer (e.g., at least five years, at least six years, at least seven years, at least eight years, at least nine years, at least ten years, or more than ten years, such as for the lifetime of the patient). Moreover, the sALP (e.g., SEQ ID NO: 1) may be administered at a dosage of about 1.3 mg/kg/week, about 2.7 mg/kg/week, or about 6 mg/kg/week or more (e.g., about 9 mg/kg/week), such as the sALP is administered at a dosage of about 2 mg/kg three times a week, about 3 mg/kg two times a week, about 3 mg/kg three times a week, or about 1 mg/kg six times a week. Additionally, the sALP may be administered once daily on consecutive or alternating days.

The method can further include: (i) increasing a dose or frequency of administration of the sALP if the patient exhibits a decrease of one or more health state (e.g., the health state is I, II, III, or IV), or does not exhibit an improvement of at least one health state, in the health state after treatment with the sALP; (ii) maintaining a dose or frequency of administration of the sALP if the patient exhibits the same health state or an improvement of at least one health state after treatment with the sALP; or (iii) reducing a dose or frequency of administration of the sALP if the patient exhibits an improvement of more than one health state after treatment with the sALP. The sALP (e.g., SEQ ID NO: 1) can be administered in a composition including at least one pharmaceutically acceptable carrier, diluent, or excipient, such as saline or sodium chloride and sodium phosphate. For example, at least one pharmaceutically acceptable carrier, diluent, or excipient includes 150 mM sodium chloride and 25 mM sodium phosphate. Moreover, the pharmaceutical composition may be administered subcutaneously, intramuscularly, intravenously, orally, nasally, sublingually, intrathecally, or intradermally. In particular, the pharmaceutical composition is administered subcutaneously.

In any of the above aspects, the sALP (e.g., SEQ ID NO: 1) includes or consists of the amino acid sequence of SEQ ID NO: 1. For example, the sALP (e.g., SEQ ID NO: 1) is physiologically active toward PEA, PPi, and PLP, catalytically competent to improve skeletal mineralization in bone, and/or is the soluble extracellular domain of an alkaline phosphatase. The sALP in the composition is, e.g., a dimer.

The patient with HPP (e.g., a child having HPP of about 5 years of age to about 12 years of age, an adolescent having HPP of about 13 years of age to about 17 years of age, or an adult having HPP of greater than about 18 years of age or older) may be a naïve patient. In particular, a HPP patient is a human.

### Definitions

As used herein, “a” and “an” means “at least one” and “one or more” unless otherwise indicated. In addition, the singular forms “a”, “an”, and “the” include plural referents unless the context clearly dictates otherwise.

As used herein, “about” refers to an amount that is ±10% of the recited value and is preferably ±5% of the recited value, or more preferably ±2% of the recited value.

By “asfotase alfa” is meant a human TNALP (hTNALP) fusion protein formulated for the treatment of HPP. Asfotase alfa (STRENSIQ®, Alexion Pharmaceuticals, Inc.) is a fusion protein including a soluble glycoprotein of two identical polypeptide chains, in which each polypeptide chain includes amino acid residues 1-726 of SEQ ID NO: 1. The structure of each polypeptide chain includes the catalytic domain of hTNALP, the human immunoglobulin Gi Fc domain, and a deca-aspartate peptide used as a bone targeting domain (the structure hTNALP-Fc-D₁₀). The two polypeptide chains are covalently linked by two disulfide bonds. Asfotase alfa has been approved throughout the world under the trade name STRENSIQ®, including in the United States, Europe, Japan, Canada, Israel, Australia, and Korea.

As used herein, “average” refers to a numerical value expressing the mean or median of a data set. The mean of a data set is calculated by dividing the sum of the values in the set by their number. The median of a date set is calculated by determining the middle value in a list of odd numbers or by determining the mean of the two data values in the middle in a list of even numbers.

The term “bone-targeting moiety,” as used herein, refers to an amino acid sequence of between 1 and 50 amino acid residues in length having a sufficient affinity to the bone matrix, such that the bone-targeting moiety, singularly, has an in vivo binding affinity to the bone matrix that is about 10⁻⁶ M to about 10⁻¹⁵ M (e.g., 10⁻⁷ M, 10⁻⁸ M, 10⁻⁹ M, 10⁻¹⁰ M, 10⁻¹¹ M, 10⁻¹² M, 10⁻¹³ M, 10⁻¹⁴ M, or 10⁻¹⁵ M).

The terms “Bruininks-Oseretsky Test of Motor Proficiency 2nd Edition” and “BOT-2,” as used herein, refer to the second edition of a standardized test of gross and fine motor performance for a patient having HPP, e.g., a child having HPP of about 5 years of age to about 12 years of age, an adolescent having HPP of about 13 years of age to about 17 years of age, or an adult having HPP of greater than about 18 years of age or older. See Bruininks, R. H. (2005). Bruininks-Oseretsky Test of Motor Proficiency, (BOT-2). Minneapolis, Minn.: Pearson Assessment, hereby incorporated by reference in its entirety. The BOT-2 is administered individually to assess gross and fine motor skills of a range of patients. The BOT-2, for example, can be used to evaluate physical impairments and mobility restrictions in patients having HPP, e.g., children having HPP of about 5 years of age to about 12 years of age, adolescents having HPP of about 13 years of age to about 17 years of age, or adults having HPP of greater than about 18 years of age or older. The BOT-2 provides composite BOT-2 scores in the following exemplary areas: strength, running speed and agility, fine motor precision, fine motor integration, manual dexterity, bilateral coordination, balance, and upper-limb coordination. For example, a BOT-2 strength total score can be determined by having a patient perform sit-ups, v-ups, standing long jump, wall sit, and push-ups. A running speed and agility total score can be determined by having a patient step over a balance beam or perform a shuttle run, two-legged side hop, or one-legged side hop. Both BOT-2 total strength and BOT-2 running speed and agility total scores range from 0 to 25, in which a score of about 10 to 25 is considered representative of healthy subjects.

The term “catalytically competent,” as used herein, refers to an sALP that hydrolyzes the bone mineralization inhibitor inorganic pyrophosphate (PPi) to provide inorganic phosphate (Pi), thereby decreasing the extracellular concentrations of PPi. Thus, the catalytically competent sALP improves skeletal mineralization in bone by regulating the concentration of PPi.

The terms “Childhood Health Assessment Questionnaire” and “CHAQ,” as used herein refer to a questionnaire that is used to assess the health state (e.g., ability to perform activities of daily living (ADLs) and incidence of pain) of patients of 1 to 19 years of age, such as children, adolescents, and some adults with HPP. For a description of the CHAQ index, see Bruce & Fries (J. Rheumatol. 30(1): 167-178, 2003), hereby incorporated by reference in its entirety. The CHAQ may be administered by interview or self-report for children greater than 8 years of age. The CHAQ includes eight sub-scales for dressing/grooming, arising, eating, walking, hygiene, reach, grip, and activities. The range of scores within each category is from 0 to 3, in which a score of 0 indicates without any difficulty; a score of 1 indicates with some difficulty; a score of 2 indicates with much difficulty; and a score of 3 indicates that the patient is unable to perform the activity. The CHAQ index may also be used to determine the presence and severity of pain.

The terms “EuroQol five dimension questionnaire” and “EQ-5D,” as used herein, refer to a questionnaire that is used to assess the health state (e.g., mobility, self-care, ability to perform usual activities of school, work, or housework, ability to perform ADLs (e.g., dressing, toileting, and cooking), experience of pain or discomfort, and anxiety or depression) of patients, such as children having HPP of about 5 years of age to about 12 years of age, adolescents having HPP of about 13 years of age to about 17 years of age, or adults having HPP of greater than about 18 years of age or older. For a description of the EQ-5D index, see Reenan & Oppe (EQ-5D-3L User Guide Version 5.1, 2015), hereby incorporated by reference in its entirety. The EQ-5D may be self-administered, or administered by a clinician or in an interview. The EQ-5D questionnaire includes the following five dimensions that characterize the health state of the HPP patient: mobility, self-care, ability to perform ADLs, incidence of pain or discomfort, and anxiety or depression. As described herein, the EQ-5D may be used in combination with at least one physical assessment, such as the 6MWT, to categorize an HPP patient as having a health state of level I indicating no problems with physiological condition, level II indicating some problems with physiological condition, level III indicating extreme problems with physiological condition, or level IV indicating the most extreme problems of physiological condition. The EQ-5D can also be used as part of the analysis to assess the transition of an HPP patient from one health state to another health state, such as from a health state of IV to III, IV to II, IV to I, III to II, III to I, or II to I. The Child Health Utility Index -9D (CHU-9D) can also be used to assess health status in HPP patients. For a description of the CHU-9D and EQ-5D indices, see Stevens (Appl Health Econ Health Policy. 9(3): 157-69, 2011), hereby incorporated by reference in its entirety.

By “extracellular domain” is meant any functional extracellular portion of the native protein, e.g., alkaline phosphatase. In particular, the extracellular domain lacks the signal peptide.

By “Fc” is meant a fragment crystallizable region of an immunoglobulin, e.g., IgG-1, IgG-2, IgG-3, IgG-3 or IgG-4, including the CH2 and CH3 domains of the immunoglobulin heavy chain. Fc may also include any portion of the hinge region joining the Fab and Fc regions. The Fc can be of any mammal, including human, and may be post-translationally modified (e.g., by glycosylation). In a non-limiting example, Fc can be the fragment crystallizable region of human IgG-1 having the amino acid sequence of SEQ ID NO: 20.

By “fragment” is meant a portion of a polypeptide or nucleic acid molecule that contains, preferably, at least 10%, 20%, 30%, 40%, 50%, 60%, 70%, 75%, 80%, 85%, 90%, 95%, 96%, 97%, 98%, 99%, or more of the entire length of the reference nucleic acid molecule or polypeptide. A fragment may contain, e.g., 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 400, 500, 600, 700, or more amino acid residues, up to the entire length of the polypeptide. Exemplary sALP fragments have amino acid residues 18-498, 18-499, 18-500, 18-501, 18-502, 18-503, 18-504, 18-505, 18-506, 18-507, 18-508, 18-509, 18-510, 18-511, or 18-512 of a ALP (e.g., SEQ ID NOs: 2-6), and may include additional C-terminal and/or N-terminal portions.

The term “health state,” as used herein, refers to the characterized physiological condition of a patient having HPP, such as a child having HPP of about 5 years of age to about 12 years of age, an adolescent having HPP of about 13 years of age to about 17 years of age, or an adult having HPP of greater than about 18 years of age or older. The health state of the HPP patient can be characterized with at least one physical assessment selected from one or more of the following metrics: 6MWT, BOT-2, BSID-III, and gait analysis, and at least one quality of life assessment selected from one or more of the following metrics: EQ-5D, CHAQ, PODCI, CHU-9D, SF-36, SF-12, and PedsQL. In particular, the health state of the HPP patient is characterized by, e.g., the 6MWT in combination with the EQ-5D. After obtaining the results of at least one physical assessment and at least one quality of life assessment selected from the above metrics, the HPP patient may be identified as having a health state of level I indicating no problems with physiological condition, level II indicating some problems with physiological condition, level III indicating extreme problems with physiological condition, or level IV indicating the most extreme problems of physiological condition. The metric(s) can be used to assess transition of the HPP patient from one health state to another health state after, e.g., treatment with an sALP as described herein (e.g., the sALP polypeptide of SEQ ID NO: 1 or a polypeptide variant having at least 95% sequence identity to the sequence of SEQ ID NO: 1, e.g., asfotase alfa), such as a transitions from a health state of IV to III, IV to II, IV to I, III to II, III to I, or II to I after administration of the sALP. The terms “hypophosphatasia” and “HPP,” as used herein, refer to a rare, heritable skeletal disorder caused by, e.g., one or more loss-of-function mutations in the ALPL (alkaline phosphatase, liver/bone/kidney) gene, which encodes tissue-nonspecific alkaline phosphatase (TNALP). HPP may be further characterized as infantile HPP, childhood HPP, perinatal HPP (e.g., benign perinatal HPP or lethal perinatal HPP), odonto-HPP, adolescent HPP, or adult HPP. For instance, “childhood HPP describes a patient having HPP that is about 5 years of age to about 12 years, “adolescent HPP” describes a patient having HPP that is about 13 years of age to about 17 years, and “adult HPP” describes a patient having HPP that is about 18 years of age or older. The term “adult HPP,” as used herein, refers to a condition or phenotype characterized by the presence of one or more of the following symptoms: elevated blood and/or urine levels of inorganic pyrophosphate (PPi), hypomineralization, hypercalciuria, one or more skeletal deformities, hypotonia, muscle weakness, rheumatoid complications, waddling gait, ambulatory difficulties, bone pain, pain, bone fracture, calcium pyrophosphate dihydrate crystal deposition, pseudogout, arthritis, pyrophosphate arthropathy, chondrocalcinosis, calcific periarthritis, and pseudofracture. The term “adolescent HPP,” as used herein, refers to a condition or phenotype characterized by the presence of one or more of the following symptoms: elevated blood or urine levels of PPi, PEA, or PLP; osteomalacia, one or more skeletal deformities, hypotonia, muscle weakness, rheumatoid complications, arthritis, pseudogout, waddling gait, ambulatory difficulties, bone pain, pain, premature loss of teeth, hypomineralization, pulmonary hypoplasia, respiratory insufficiency, seizures, hypercalciuria, short stature, and growth delay. The term “childhood HPP,” as used herein, refers to refers to a condition or phenotype characterized by the presence of one or more of the following symptoms: elevated blood or urine levels of PPi, PEA, or PLP; rickets, rachitic ribs, one or more skeletal deformities, hypotonia, muscle weakness, rheumatoid complications, arthritis, pseudogout, waddling gait, ambulatory difficulties, bone pain, pain, premature loss of teeth, hypomineralization, delayed motor development, seizures, hypercalciuria, short stature, bone fracture, pseudofracture, and growth delay.

By “naïve patient” and “naïve subject” is meant a patient or subject having HPP (e.g., a child having HPP of about 5 years of age to about 12 years of age, an adolescent having HPP of about 13 years of age to about 17 years of age, or an adult having HPP of greater than about 18 years of age or older) that has not previously received treatment with an alkaline phosphatase, or a polypeptide having alkaline phosphatase activity, such as an sALP (e.g., TNALP, for example the sALP polypeptide of SEQ ID NO: 1 or a polypeptide variant having at least 95% sequence identity to the sequence of SEQ ID NO: 1, e.g., asfotase alfa).

The terms “polypeptide” and “protein” are used interchangeably and refer to any chain of two or more natural or unnatural amino acid residues, regardless of post-translational modification (e.g., glycosylation or phosphorylation), constituting all or part of a naturally-occurring or non-naturally occurring polypeptide or peptide, as is described herein.

By “pharmaceutically acceptable carrier, diluent, or excipient” is meant a carrier, diluent, or excipient, respectively, that is physiologically acceptable to the subject (e.g., a human) while retaining the therapeutic properties of the pharmaceutical composition with which it is administered. One exemplary pharmaceutically acceptable carrier, diluent, or excipient is physiological saline. For instance, the pharmaceutically acceptable carrier, diluent, or excipient can include sodium chloride (e.g., 150 mM sodium chloride) and sodium phosphate (e.g., 25 mM sodium phosphate). Other physiologically acceptable carriers, diluents, or excipients and their formulations are known to one skilled in the art.

By “pharmaceutical composition” is meant a composition containing a polypeptide (e.g., compositions including an sALP, such as asfotase alfa) as described herein formulated with at least one pharmaceutically acceptable carrier, diluent, or excipient. The pharmaceutical composition may be manufactured or sold with the approval of a governmental regulatory agency as part of a therapeutic regimen for the treatment or prevention of a disease or event in a patient. Pharmaceutical compositions can be formulated, for example, for subcutaneous administration, intravenous administration (e.g., as a sterile solution free of particulate emboli and in a solvent system suitable for intravenous use), for oral administration (e.g., a tablet, capsule, caplet, gelcap, or syrup), or any other formulation described herein, e.g., in unit dosage form.

The term “physiological condition,” as used herein, refers to one or more symptoms, such as bone weakness and muscle weakness, associated with HPP that can restrict or eliminate, e.g., walking ability, functional endurance, and ability to perform activities of daily living (ADL) of a HPP patient (e.g., a child having HPP of about 5 years of age to about 12 years of age, an adolescent having HPP of about 13 years of age to about 17 years of age, or an adult having HPP of greater than about 18 years of age or older). The physiological condition of the HPP patient may be characterized with at least one physical assessment, particularly selected from the following metrics: Six Minute Walk Test (6MWT), Bruininks-Oseretsky Test of Motor Proficiency 2nd Edition (BOT-2), Bayley Scales of Infant and Toddler Development, 3rd Edition (BSID-III), or gait analysis, and at least one quality of life assessment, particularly selected from the following metrics: EuroQol five dimension questionnaire (EQ-5D), Childhood Health Assessment Questionnaire (CHAQ), Child Health Utility Index -9D (CHU-9D), Pediatric Quality of Life Inventory (PedsQL), Pediatric Outcomes Data Collection Instrument (PODCI), Short Form Health Survey 36 (SF-36), or Short Form Health Survey 12 (SF-12), as described herein. In particular, the physiological condition of an HPP patient may restrict or eliminate a patient's ability to perform ADL, which are routine activities that healthy subjects perform on a daily basis without requiring assistance, such as functional mobility or transferring (e.g., walking), bathing and showering, dressing, self-feeding, and personal hygiene and grooming. As described herein, therapeutic compositions (e.g., compositions including an sALP, such as asfotase alfa) can be administered to a patient (e.g., a child having HPP of about 5 years of age to about 12 years of age, an adolescent having HPP of about 13 years of age to about 17 years of age, or an adult having HPP of greater than about 18 years of age or older) to decrease the severity and/or frequency of physiological conditions associated with an HPP phenotype.

The terms “Pediatric Outcomes Data Collection Instrument” and “PODCI,” as used herein, refer to a questionnaire used to assess overall health, incidence of pain, and ability to perform ADLs of patients under 19 years of age, particularly in patients with chronic health disorders, such as patients with HPP. For a description of the PODCI, see Plint et al. (J. Pediatr. Orthop. 23(6): 788-790, 2003), hereby incorporated by reference in its entirety. The questionnaire may be completed by the patient or by a parent/guardian of the patient with knowledge of the patient's condition. The eight scales generated from the PODCI include the following: 1) the upper extremity and physical function scale to measure difficulty encountered in performing daily personal care and student activities; 2) the transfer and basic mobility scale to measure difficulty experienced in performing routine motion and motor activities in daily activities; 3) the sports/physical functioning scale to measure difficulty or limitations encountered in participating in more active activities or sports; 4) the pain/comfort scale to measure the level of pain experienced during the past week; 5) the treatment expectations scale to measure the long term expectations of treatment; 6) the happiness scale to measure overall satisfaction with personal looks and sense of similarity to friends and others of own age; 7) the satisfaction with symptoms scale to measure the patient's acceptance of current limitations should this be a life-long state; and 8) the global functioning scale, which is a general combined scale calculated from the first four scales listed above. Standardized scores are generated from a series of questions in the PODCI and converted to a 0 to 100 scale, in which 0 represents significant disability and 100 represents less disability.

The term “physiologically active,” as used herein, refers to an sALP (e.g., SEQ ID NO: 1) that hydrolyzes phosphoethanolamine (PEA), inorganic pyrophosphate (PPi), and pyridoxal 5′-phosphate (PLP) to provide Pi, thereby decreasing extracellular concentrations of PEA, PPi, and PLP.

The terms “sALP,” “soluble alkaline phosphatase,” and “extracellular domain of an alkaline phosphatase” are used interchangeably and refer to a soluble, non-membrane-bound alkaline phosphatase or a domain, biologically active fragment, or biologically active variant thereof. sALPs include, for example, an alkaline phosphatase lacking a C-terminal glycolipid anchor (GPI signal sequence, e.g., polypeptides including or consisting of the amino acid residues 18-502 of a human TNALP (SEQ ID NOs: 2, 3, 4, 5, or 6)). In particular, a TNALP may include, e.g., a polypeptide including or consisting of amino acid residues 1-485 of SEQ ID NO: 1, such as asfotase alfa, or a polypeptide variant having at least 95% sequence identity to the amino acid residues 1-485 of SEQ ID NO: 1. sALPs further include, for example, mammalian orthologs of human TNALP, such as a rhesus TNALP (SEQ ID NO: 7), a rat TNALP (SEQ ID NO: 8), a canine TNALP (SEQ ID NO: 9), a porcine TNALP (SEQ ID NO: 10), a murine TNALP (SEQ ID NO: 11), a bovine TNALP (SEQ ID NOs: 12-14), or a feline TNALP (SEQ ID NO: 15). sALPs also include soluble, non-membrane-bound forms of human PALP (e.g., polypeptides including or consisting of amino acid residues 18-502 of SEQ ID NOs: 16 or 17), GCALP (e.g., polypeptides including or consisting of amino acid residues 18-502 of SEQ ID NO: 18), and IALP (e.g., polypeptides including or consisting of amino acid residues 18-502 of SEQ ID NO: 19), and additional variants and analogs thereof that retain alkaline phosphatase activity, e.g., the ability to hydrolyze PPi.

An sALP, in particular, lacks the N-terminal signal peptide (e.g., aa 1-17 of SEQ ID NOs: 2-6, 8, 11-13, or 15 or aa 1-25 of SEQ ID NO: 7).

By “sALP polypeptide” is meant a polypeptide having the structure A-sALP-B, wherein sALP is as defined herein and each of A and B is absent or is an amino acid sequence of at least one amino acid (e.g., any sALP fusion polypeptide described herein (for example the sALP fusion polypeptide of SEQ ID NO: 1 or a polypeptide variant having at least 95% sequence identity to the sequence of SEQ ID NO: 1, e.g., asfotase alfa).

By “signal peptide” is meant a short peptide (5-30 amino acids long) at the N-terminus of a polypeptide that directs a polypeptide towards the secretory pathway (e.g., the extracellular space). The signal peptide is typically cleaved during secretion of the polypeptide. The signal sequence may direct the polypeptide to an intracellular compartment or organelle, e.g., the Golgi apparatus. A signal sequence may be identified by homology, or biological activity, to a peptide with the known function of targeting a polypeptide to a particular region of the cell. One of ordinary skill in the art can identify a signal peptide by using readily available software (e.g., Sequence Analysis Software Package of the Genetics Computer Group, University of Wisconsin Biotechnology Center, 1710 University Avenue, Madison, Wis. 53705, BLAST, or PILEUP/PRETTYBOX programs). A signal peptide can be one that is, for example, substantially identical to amino acid residues 1-17 of SEQ ID NOs: 2-6 or amino acid residues 1-25 of SEQ ID NO: 7.

As used herein, when a polypeptide or nucleic acid sequence is referred to as having “at least X% sequence identity” to a reference sequence, wherein “X” is a real number, it is meant that at least X percent of the amino acid residues or nucleotides in the polypeptide or nucleic acid are identical to those of the reference sequence when the sequences are optimally aligned. An optimal alignment of sequences can be determined in various ways that are within the skill in the art, for instance, the Smith Waterman alignment algorithm (Smith et al., J. Mol. Biol. 147:195-7, 1981) and BLAST (Basic Local Alignment Search Tool; Altschul et al., J. Mol. Biol. 215: 403-10, 1990). These and other alignment algorithms are accessible using publicly available computer software such as “Best Fit” (Smith and Waterman, Advances in Applied Mathematics, 482-489, 1981) as incorporated into GeneMatcher Plus (Schwarz and Dayhoff, Atlas of Protein Sequence and Structure, Dayhoff, M. O., Ed. pp 353-358, 1979), BLAST, BLAST-2, BLAST-P, BLAST-N, BLAST-X, WU-BLAST-2, ALIGN, ALIGN-2, CLUSTAL, Megalign (DNASTAR), or other software/hardware for alignment. In addition, those skilled in the art can determine appropriate parameters for measuring alignment, including any algorithms needed to achieve optimal alignment over the length of the sequences being compared.

The terms “patient” and “subject” refer to a mammal, including, but not limited to, a human or a non-human mammal, such as a bovine, equine, canine, ovine, or feline. Of particular interest are human patients.

“Parenteral administration,” “administered parenterally,” and other grammatically equivalent phrases, as used herein, refer to modes of administration other than enteral and topical administration, usually by injection, and include, without limitation, subcutaneous, intradermal, intravenous, intranasal, intraocular, pulmonary, intramuscular, intra-arterial, intrathecal, intracapsular, intraorbital, intracardiac, intradermal, intrapulmonary, intraperitoneal, transtracheal, subcuticular, intraarticular, subcapsular, subarachnoid, intraspinal, epidural, intracerebral, intracranial, intracarotid, and intrasternal injection and infusion.

As used herein, “Six Minute Walk Test” and “6MWT” refer to a physical assessment that is a standardized test to assess walking ability of a patient having HPP (e.g., a child having HPP of about 5 years of age to about 12 years of age, an adolescent having HPP of about 13 years of age to about 17 years of age, or an adult having HPP of greater than about 18 years of age or older). In particular, walking ability refers to the ability of the patient to lift and set down each foot in turn. See the American Thoracic Society statement: guidelines for the six-minute walk test (Amer. J. of Respiratory and Critical Care Medicine, 166(1):111-7, 2002, hereby incorporated by reference in its entirety). The 6MWT is determined from the distance (e.g., in meters) that a patient walks on a flat, hard surface in a period of six minutes. The 6MWT distance can then be compared to the 6MWT distance of the patient at baseline, the 6MWT distance of an untreated subject (e.g., an untreated subject of about the same age, height, and/or gender), or the 6MWT distance of a healthy subject (e.g., a healthy subject of about the same age, height, and/or gender) and expressed as a percentage to determine the 6MWT value.

By “treating,” “treat,” and “treatment” is meant the medical management of a patient with the intent to cure, ameliorate, stabilize, reduce the likelihood of, or prevent HPP (e.g., child, adolescent, or adult HPP) and/or management of a patient exhibiting or likely to have HPP, e.g., by administering a pharmaceutical composition (e.g., an sALP, such as SEQ ID NO: 1). This term includes active treatment, that is, treatment directed specifically toward the improvement or associated with the cure of a disease, pathological condition, disorder, or event, and also includes causal treatment, that is, treatment directed toward removal of the cause of the associated disease, pathological condition, disorder, or event. In addition, this term includes palliative treatment, that is, treatment designed for the relief or improvement of at least one symptom rather than the curing of the disease, pathological condition, disorder, or event; symptomatic treatment, that is, treatment directed toward constitutional symptoms of the associated disease, pathological condition, disorder, or event; preventative treatment, that is, treatment directed to minimizing or partially or completely inhibiting the development of the associated disease, pathological condition, disorder, or event, e.g., in a patient who is not yet ill, but who is susceptible to, or otherwise at risk of, a particular disease, pathological condition, disorder, or event; and supportive treatment, that is, treatment employed to supplement another specific therapy directed toward the improvement of the associated disease, pathological condition, disorder, or event.

Other features and advantages of the present disclosure will be apparent from the following Detailed Description and claims.

### BRIEF DESCRIPTION OF THE DRAWINGS

FIGS. 1A-1C are bar graphs showing patient distribution across HPP health states at baseline and after 96 weeks of asfotase alfa treatment in patients aged 5-12 years (FIG. 1A), 13-17 years (FIG. 1B), and 18 years (FIG. 10).

FIG. 2 is a bar graph showing the transition of patients between HPP health states after 96 weeks of treatment with asfotase alfa.

FIG. 3 is a bar graph showing the median percentage predicted 6MWT results at baseline and after 96 weeks of asfotase alfa treatment.

FIG. 4 is a graph showing key health characteristics (respiratory function, fractures, dental complications, mobility/strength/agility, pain, and independence) of 12 different patient profiles, which are composed of pre-adolescents (age 5-12), adolescents (age 13-17), and adults (age 18+) in each of four health states (I, II, III, and IV).

FIG. 5A is a bar graph of clinical symptoms and events over time for cases with first HPP symptom identified in infancy/early childhood, from 0 to <6 months (n=48).

FIG. 5B is a bar graph of clinical symptoms and events over time for cases with first HPP symptom identified in infancy/early childhood, from 6 to <24 months (n=52).

FIGS. 6A-6E are a set of timelines showing median (range) time to HRQoL impacting symptoms by age at disease onset in utero (FIG. 6A), infancy (FIG. 6B), childhood (FIG. 6C), adolescence (FIG. 6D), and adulthood (FIG. 6E).

FIG. 7 is a timeline of median (range) time to HRQoL impacting symptoms for n-265 cases of HPP

FIGS. 8A-8G are a set of Kaplan-Meier curves for time to premature loss of teeth (FIG. 8A), fracture (FIG. 8B), gross motor or ambulation difficulties (FIG. 8C), pain (FIG. 8D), surgery (FIG. 8E), cranial abnormalities (FIG. 8F), and respiratory symptoms (FIG. 8G).

### DETAILED DESCRIPTION

It has been discovered that hypophosphatasia (HPP) patients can be characterized as having different health states (e.g., a health state of I, II, III, or IV) and that these health states can be used, e.g., to monitor or identify the status of the patient, to assess therapeutic efficacy of a treatment regimen, to assess the cost effectiveness of a treatment regimen, and/or to modify or assign a treatment regimen, in particular, a treatment regimen featuring the administration of asfotase alfa (SEQ ID NO: 1, STRENSIQ®, Alexion Pharmaceuticals, Inc.) to the HPP patient. The health state assessment and assignment may differ depending upon the age of the HPP patient.

For example, the physiological condition of a child having HPP of about 5 years of age to about 12 years of age, an adolescent having HPP of about 13 years of age to about 17 years of age, or an adult having HPP of greater than about 18 years of age or older can be characterized using one or more symptoms of HPP and/or one or more metrics (e.g., Six Minute Walk Test (6MWT), EuroQol Five Dimension Questionnaire (EQ-5D), Bruininks-Oseretsky Test of Motor Proficiency 2nd Edition (BOT-2), Childhood Health Assessment Questionnaire (CHAQ), Pediatric Outcomes Data Collection Instrument (PODCI), Child Health Utility Index-9D (CHU-9D), Pediatric Quality of Life Inventory (PedsQL), Bayley Scales of Infant and Toddler Development, 3rd Edition (BSID-III), Short Form Health Survey 36 (SF-36), gait analysis, and Short Form Health Survey 12 (SF-12)) in order to determine the health state of the HPP patient, which can be assigned a health state level of I (e.g., indicating no problems with physiological condition), II (e.g., indicating some problems with physiological condition), III (e.g., indicating extreme problems with physiological condition), or IV (e.g., indicating the most extreme problems with physiological condition). For a description of the PedsQL, see Varni et al. (Med Care. 39(8):800-12, 2001), for a description of the SF-12, see Ware et al. (Med Care. 34(3):220-33, 1996), for a description of the SF-36, see Ware et al. (Med Care. 30:473-483, 1992), and for a description of BSID-III, see Bayley et al. (Bayley Scales of Infant and Toddler Development—Third Edition: Administration Manual. 2006) and Albers et al. (J. Psychoeducational Assessment, 25(2), 180-190, 2007), each of which is hereby incorporated by reference in its entirety. In particular, the health state of the HPP patient is characterized by, e.g., the 6MWT, which assesses the HPP patient's walking ability, in combination with the EQ-5D, which assesses the patient's mobility, self-care, ability to perform activities of daily living (ADLs), incidence of pain or discomfort, and anxiety or depression. After using the symptoms of HPP and/or the one or more metrics to identify the health state of the HPP patient, a treatment regimen may then be assigned to the patient based on the health state, such as a treatment regimen of administering at least 0.5 mg/kg/week (e.g., about 1 mg/kg/week to about 9 mg/kg/week, preferably 6 mg/kg/week) of a soluble alkaline phosphatase (sALP; e.g., the sALP polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least 3 months or more, such as at least 96 weeks, or for the life of the patient).

The methods can also be used to assess transition of an HPP patient (e.g., a child, adult, or adolescent HPP patient) from one health state to another. In particular, the physiological condition of the HPP patient can be characterized using at least one physical assessment (selected from, e.g., 6MWT, BOT-2, and gait analysis) and at least one quality of life assessment (selected from, e.g., EQ-5D, CHAQ, PODCI, CHU-9D, PedsQL, SF-36, and SF-12) evaluating one or more symptoms and/or one or more metrics, followed by identification of the health state of the HPP patient prior to or during administration of a treatment regimen, such as administration of at least 0.5 mg/kg/week (e.g., about 1 mg/kg/week to about 9 mg/kg/week, preferably 6 mg/kg/week) of an sALP (e.g., the sALP polypeptide of SEQ ID NO: 1 or a polypeptide variant having at least 95% sequence identity to the sequence of SEQ ID NO: 1, e.g., asfotase alfa) to the patient for a treatment period of at least two weeks. After completion of a treatment regimen, characterization of the physiological condition and identification of the health state of the HPP patient may be repeated (e.g., one or more times) to assess a change in the health state of the HPP patient (e.g., a transition from a health state of IV to III, IV to II, IV to I, III to II, III to I, or II to I after the treatment regimen featuring administration of the sALP).

For example, if the HPP patient exhibits a decrease of one or more health state levels or does not exhibit an improvement of at least one health state level after treatment with the sALP (e.g., from IV to III, IV to II, IV to I, III to II, III to I, or II to I), then the dose and/or frequency of sALP administration in the treatment regimen can be increased (e.g., an initial dosage of the sALP of about 2.1 mg/kg/week to about 3.5 mg/kg/week of the sALP may be increased to a dosage of about 6 mg/kg/week to about 9 mg/kg/week for a treatment period of at least two weeks). Alternatively, if the patient exhibits the same health state level (e.g., I, II, III, or IV) or an improvement of at least one health state level after treatment with the sALP (e.g., from IV to III, IV to II, IV to I, III to II, III to I, or II to I), then the dose and/or frequency of sALP administration in the treatment regimen can be maintained at the current dose and/or frequency of administration of the sALP, such as a dose of at least 0.5 mg/kg/week (e.g., about 1 mg/kg/week to about 9 mg/kg/week, preferably 6 mg/kg/week) of the sALP to the patient. Additionally, if the patient exhibits an improvement of more than one health state level after treatment with the sALP, then the dose and/or frequency of sALP administration in the treatment regimen can be reduced, if desired, from an initial dosage of about 6 mg/kg/week to about 9 mg/kg/week to a dosage of less than about 6 mg/kg/week, such as administration of about 2.1 mg/kg/week to about 3.5 mg/kg/week of the sALP to the patient.

### Methods of Identification and Treatment

Provided herein are methods of identifying the health state of a patient having HPP (e.g., a child having HPP of about 5 years of age to about 12 years of age, an adolescent having HPP of about 13 years of age to about 17 years of age, or an adult having HPP of greater than about 18 years of age or older) prior to initiation of, during, or after a treatment regimen involving administration of an sALP to the HPP patient. The health status, once determined, may be used, e.g., to monitor the status of the patient, to assess therapeutic efficacy of a treatment regimen, and/or to modify or assign a treatment regimen, in particular, one described herein featuring the administration of sALP (e.g., the sALP polypeptide of SEQ ID NO: 1 or a polypeptide variant having at least 95% sequence identity to the sequence of SEQ ID NO: 1, e.g., asfotase alfa) to the HPP patient. In particular, HPP patients across a range of ages can be identified based on their health state, such as a patient of, e.g., about 5 to about 7 years of age, about 6 to about 9 years of age, about 7 to about 11 years of age, about 8 to about 12 years of age, about 5 to about 10 years of age, about 9 to about 12 years of age, about 8 to about 11 years of age, about 7 to about 10 years of age, about 13 to 15 years of age, about 13 to 16 years of age, about 14 to 16 years of age, about 15 to 17 years of age, about 14 to 17 years of age, about 12 to 16 years of age, about 12 to 17 years of age, about 12 to 15 years of age, about 18 to about 20 years of age, about 20 to about 25 years of age, about 25 to about 30 years of age, about 30 to about 35 years of age, about 35 to about 40 years of age, about 40 to about 45 years of age, about 45 to about 50 years of age, about 50 to about 55 years of age, about 60 to about 65 years of age, about 20 to about 30 years of age, about 30 to about 40 years of age, about 40 to about 50 years of age, about 50 to about 60 years of age, about 60 to about 70 years of age, about 20 to about 65 years of age, about 30 to about 65, years of age, or older than 65 years of age. HPP patients can be diagnosed with HPP prior to assigning a treatment regimen based on the health state (e.g., a health state of I, II, III, or IV) of the HPP patient. The HPP patient can also be a naïve patient that has not previously received treatment with an sALP (such as TNALP, for example, an sALP fusion polypeptide, such as the sALP fusion polypeptide of SEQ ID NO: 1 or a polypeptide variant having at least 95% sequence identity to the sequence of SEQ ID NO: 1, e.g., asfotase alfa) prior to a determination of their health state (e.g., a health state of I, II, III, or IV). The health state, once determined, can be used, e.g., to assign a treatment regimen to the patient.

The method involves characterizing the physiological condition of a patient having HPP (e.g., a child having HPP of about 5 years of age to about 12 years of age, an adolescent having HPP of about 13 years of age to about 17 years of age, or an adult having HPP of greater than about 18 years of age or older) using one or more symptoms of HPP (see, e.g., Table 1 below) and/or one or more metrics, in particular, one or more of the 6MWT, EQ-5D, BOT-2, CHAQ, gait analysis, PODCI, CHU-9D, and PedsQL. The symptoms of HPP and/or the one or more metrics can be used to assign a health state to the HPP patient. The health state of the HPP patient can then be used to assign a treatment regimen to the patient, in which the treatment regimen features administration of at least 0.5 mg/kg/week of an sALP (e.g., the sALP polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). Additionally, the method can include assessing a change in the health state of the patient by repeating the characterizing step using the one or more symptoms and/or one or more metrics after completion of a treatment regimen featuring administration of at least 0.5 mg/kg/week of the sALP (e.g., a treatment regimen providing about 1 mg/kg/week to about 9 mg/kg/week of the sALP, preferably 6 mg/kg/week of the sALP) for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

Additionally, each of the described symptoms and metrics (e.g., at least one physical assessment selected from, for example, 6MWT, BOT-2, BSID-III, and gait analysis, and at least one quality of life assessment selected from, for example, EQ-5D, CHAQ, PODCI, CHU-9D, PedsQL, SF-36, and SF-12) can be used in any combination of at least one physical assessment and at least one quality of life assessment to determine the transition of a patient with HPP (e.g., a child, an adolescent, or an adult) from one health state to another after completion of a treatment regimen that includes administering an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient, in which improvements relative to a certain value or score of the metric tested can be used to show a treatment effect in the HPP patient using the sALP.

### Hypophosphatasia (HPP) By Age Group

Patients having HPP (e.g., a child having HPP of about 5 years of age to about 12 years of age, an adolescent having HPP of about 13 years of age to about 17 years of age, or an adult having HPP of greater than about 18 years of age or older) can be assigned a health state (e.g., a health state of I, II, III, or IV) using one or more symptoms (see, e.g., Table 1 below) and/or at least one physical assessment, as described herein, selected from 6MWT, BOT-2, BSID-III, and gait analysis, singly or in any combination, and at least one quality of life assessment, as described herein, selected from EQ-5D, CHAQ, PODCI, CHU-9D, PedsQL, SF-36, and SF-12, singly or in any combination. The health state of the HPP patient (e.g., a health state of I, II, III, or IV) can then be used, e.g., to assign a treatment regimen to the patient or to assess transition of the HPP patient from one health state to another after completion of a treatment regimen. After identification of the health state of the HPP patient, the patient can be assigned a treatment regimen that includes administering an sALP (such as a TNALP, for example the sALP fusion polypeptide of SEQ ID NO: 1 or a polypeptide variant having at least 95% sequence identity to the sequence of SEQ ID NO: 1, e.g., asfotase alfa) for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

In particular, asfotase alfa (STRENSIQ®) can be administered, as described herein, to treat a child having HPP of about 5 years of age to about 12 years of age, an adolescent having HPP of about 13 years of age to about 17 years of age, or an adult having HPP of greater than about 18 years of age or older (e.g., a naïve patient). Accordingly, the methods are useful for identifying and alleviating one or more, or all, of the symptoms of HPP described herein, particularly when the sALP (such as TNALP, for example the sALP fusion polypeptide of SEQ ID NO: 1 or a polypeptide variant having at least 95% sequence identity to the sequence of SEQ ID NO: 1, e.g., asfotase alfa) is administered for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years). In particular, the treatment period is at least six weeks, e.g., at least 96 weeks.

For instance, the methods are useful for treating symptoms of childhood HPP, including, but not limited to, elevated blood and/or urine levels of PPi, PEA, or PLP, rickets, rachitic ribs, one or more skeletal deformities, hypotonia, muscle weakness, rheumatoid complications, arthritis, pseudogout, waddling gait, ambulatory difficulties, bone pain, pain, premature loss of teeth, hypomineralization, delayed motor development, seizures, hypercalciuria, short stature, bone fracture, pseudofracture, and growth delay. The methods are also useful for treating symptoms of adolescent HPP, including, but not limited to, elevated blood or urine levels of PPi, PEA, or PLP, elevated blood or urine levels of PPi, PEA, or PLP; osteomalacia, one or more skeletal deformities, hypotonia, muscle weakness, rheumatoid complications, arthritis, pseudogout, waddling gait, ambulatory difficulties, bone pain, pain, premature loss of teeth, hypomineralization, pulmonary hypoplasia, respiratory insufficiency, seizures, hypercalciuria, short stature, and growth delay. Additionally, the methods are useful for treating symptoms of adult HPP, including, but not limited to, elevated blood or urine levels of PPi, PEA, or PLP, hypomineralization, hypercalciuria, one or more skeletal deformities, hypotonia, muscle weakness, rheumatoid complications, waddling gait, ambulatory difficulties, bone pain, pain, bone fracture, calcium pyrophosphate dihydrate crystal deposition, pseudogout, arthritis, pyrophosphate arthropathy, chondrocalcinosis, calcific periarthritis, and pseudofracture.

### Hypophosphatasia (HPP) Symptoms Useful for the Identification of Health State

A patient having HPP (e.g., a child having HPP of about 5 years of age to about 12 years of age, an adolescent having HPP of about 13 years of age to about 17 years of age, or an adult having HPP of greater than about 18 years of age) may be identified as having a health state of I, II, III, or IV based one or more symptoms of HPP. One or more symptoms may be grouped into the following categories: dental health, stature, mobility, strength/muscle weakness, activities of daily living (ADL), pain, sleep, emotions, participation in school/work, social relationships, independence, respiratory function, risk of fractures, tooth loss, frequency of medical appointments, and/or craniosynostosis (Table 1).

TABLE 1
Descriptions of HPP in patients identified as health state I, II, III, and IV.

| Symptom | | | | |
|---|---|---|---|---|
| Category | Health State I | Health State II | Health State III | Health State IV |
| Dental | Patient may have a | Patient may have a | Patient may have a | Patient has |
| Health | slightly unusual | slightly unusual | slightly unusual | noticeable skeletal |
| | appearance due in | appearance due in | appearance due in | deformities and an |
| | part to their tooth | part to their tooth | part to their tooth | unusual appearance. |
| | loss. | loss. | loss. | |
| Stature | Patient has mild | Patient has mild | Patient has | Patient has |
| | short stature for | short stature for | shortened stature | shortened stature. |
| | their age. | their age. | for their age. | |
| Mobility | Patient has near | Patient has slightly | Patient has | Patient struggles to |
| | normal mobility for | reduced mobility | noticeably impaired | walk to school or |
| | their age. They may | for their age. They | mobility for their age. | work and often |
| | run awkwardly and | walk slowly and | They walk very slowly | relies upon a |
| | may be slightly | may be slightly | and awkwardly and | wheelchair or |
| | clumsy. | clumsy. | may be clumsy. | walking aids. |
| Strength/ | Patient may not be | Patient may not be | Patient has some | Patient has |
| Muscle | as strong as or have | as strong as or | muscle weakness | significant muscle |
| Weakness | the agility of an | have the agility of | and is not as strong | weakness and is |
| | otherwise healthy | an otherwise | or agile as an | significantly less |
| | person of their age. | healthy person of | otherwise healthy | agile than an |
| | | their age. | person of their age. | otherwise healthy |
| | | | | person of their age. |
| Activities | Patient is able to | Patient has mild | Patient has problems | Patient has severe |
| of Daily | complete usual | problems | completing their usual | problems with any |
| Living | activities such as | completing their | activities such as | physical activity and |
| | washing & dressing, | usual activities | dressing, going to | could not participate |
| | going to school or | such as dressing, | school or work, and | in sport. Patient has |
| | work, playing sport. | going to school or | would find it very | gross motor delay. |
| | | work, playing sport. | difficult to participate | Patient has problems |
| | | | in sports. Patient has | with washing and |
| | | | motor delay. | dressing. Patient |
| | | | Patient can wash and | has difficulties with |
| | | | dress themselves | their daily activities |
| | | | however they have some | because of severe pain, |
| | | | difficulties with their | muscle weakness and |
| | | | daily activities at | other limitations in |
| | | | times because of pain, | physical functioning. |
| | | | muscle weakness and | |
| | | | other limitations in | |
| | | | physical functioning. | |
| Pain | Patient has no | Patient has some | Patient regularly | Patient experiences |
| | chronic pain | intermittent pain | experiences pain | chronic pain that |
| | associated with their | associated with | associated with their | they receive |
| | HPP, but may | their HPP. | HPP. | medication for. |
| | experience pain | | | |
| | during or after | | | |
| | physical activities | | | |
| | such as sport. | | | |
| Sleep | Patient can sleep | Patient can sleep | Sleep may be | Sleep may be |
| | normally. | normally. | disturbed due to pain | disturbed due to |
| | | | or discomfort. | pain or discomfort. |
| Emotions | Patient's mood, | Patient's mood, | The patient's health | The patient's health |
| | anxiety or sadness | anxiety or sadness | problems have | problems have |
| | varies in the same | varies in the same | affected their self- | affected their self- |
| | way that an | way that an | esteem. They have | esteem. They have |
| | otherwise healthy | otherwise healthy | mildly impaired | diminished |
| | patient's would be | patient's would be | psychological | psychological |
| | expected to. | expected to. | wellbeing and | wellbeing and fewer |
| | | | reduced social | social contacts than |
| | | | functioning. | an otherwise healthy |
| | | | | person their age. |
| Participation | Patient can go to | Patient can go to | Patient can go to | School or work life |
| in school/ | school or work and | school or work but | school or work but | is very disrupted by |
| work | doesn't have undue | may have some | has problems | their HPP related |
| | problems with | problems with | completing tasks. | problems. They |
| | completing tasks. | completing tasks. | | sometime have to |
| | | | | spend days at home. |
| Social | Patient has the | Patient has the | Patient some | Patient is unable to |
| relationships | normal range of | normal range of | limitations to their | have a normal |
| | relationships for | relationships for | social life because of | social life because |
| | someone their age. | someone their age. | interrupted | of interrupted |
| | | | school/work life and | school/work life and |
| | | | the burden of living | the burden of living |
| | | | with HPP. | with HPP. |
| Independence | The patient has the | The patient has the | Compared to an | Compared to an |
| | same level of | same level of | otherwise healthy | otherwise healthy |
| | independence as a | independence as a | person the same age | person the same |
| | healthy person the | healthy person the | the patient | age the patient |
| | same age. | same age. | occasionally | experiences a lack |
| | | | experiences a lack of | of independence in |
| | | | independence in their | their day to day life. |
| | | | day to day life. | |
| Respiratory | Respiratory | Respiratory | Patient may | Patient may |
| function | function is normal | function is normal | experience occasional | experience prolonged |
| | or near normal. | or near normal. | respiratory problems. | respiratory infections. |
| Risk of | Patient has no | Patient has a slight | Patient has an | Patient has an |
| fractures | increased risk of | increased risk of | increased risk of | increased risk of |
| | fractures or | fractures or | fractures and | fractures and |
| | associated pain. | associated pain. | associated pain. | associated pain. |
| Tooth loss | Patient may have | Patient may have | Patient may have | Patient may have |
| | prematurely lost | prematurely lost | prematurely lost | prematurely lost |
| | several teeth and | several teeth and | several teeth and | several teeth and |
| | may have other | may have other | may have other | may have other |
| | general dental | general dental | general dental | general dental |
| | complications. | complications. | complications. | complications. |
| Frequency of | Patient has annual | Patient has annual | Patient has regular | Patient has regular |
| medical | appointments to | appointments to | appointments to | appointments to |
| appointments | review their health | review their health | review their health | review their health |
| | and check their | and check their | and check their | and check their |
| | development. | development. | development. | development. |
| Craniosynostosis | Patient is unlikely | Patient is unlikely | Patient may have a | Patient is likely to |
| | to have a history of | to have a history of | history of | have a history of |
| | craniosynostosis. | craniosynostosis. | craniosynostosis | craniosynostosis |
| | | | and may have undergone | and has undergone |
| | | | surgery on their skull | surgery on their |
| | | | previously. | skull previously. |

For example, an HPP patient (e.g., a child having HPP of about 5 years of age to about 12 years of age, an adolescent having HPP of about 13 years of age to about 17 years of age, or an adult having HPP of greater than about 18 years of age) can be assigned health state I if one or more of the following symptoms are present: a slightly unusual appearance due in part to tooth loss; mild short stature for their age; near normal mobility for their age; awkward run and may be slightly clumsy; not be as strong or lack agility of healthy subject of same age; ability to complete usual activities (e. g., washing and dressing, going to school or work, and playing sports); no chronic pain associated with HPP, but may experience pain during or after physical activities, such as sport; normal sleep; mood, anxiety or sadness varies similar to healthy subject; can attend school or work without undue problems with completing tasks; normal range of relationships for their age; same level of independence as a healthy person of same age; respiratory function is normal or near normal; no increased risk of fractures or associated pain; premature loss of several teeth or other general dental complications; and unlikely to have a history of craniosynostosis. The health state of the HPP patient can then be used as described herein to assign a treatment regimen to the patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa).

An HPP patient (e.g., a child having HPP of about 5 years of age to about 12 years of age, an adolescent having HPP of about 13 years of age to about 17 years of age, or an adult having HPP of greater than about 18 years of age) can be assigned health state II if one or more of the following symptoms are present: a slightly unusual appearance due in part to their tooth loss; mild short stature for their age; slightly reduced mobility for their age; walk slowly and may be slightly clumsy; not as strong as or lack agility of an otherwise healthy person of their age; mild problems completing usual activities, such as dressing, going to school or work, and playing sport; some intermittent pain associated with HPP; normal sleep; mood, anxiety or sadness similar to healthy subject; can attend school or work, but may have some problems with completing tasks; normal range of relationships for someone their age; same level of independence as a healthy subject the same age; respiratory function is normal or near normal; a slight increased risk of fractures or associated pain; premature loss of several teeth and other general dental complications; and unlikely to have a history of craniosynostosis. The health state of the HPP patient can then be used as described herein to assign a treatment regimen to the patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa).

An HPP patient (e.g., a child having HPP of about 5 years of age to about 12 years of age, an adolescent having HPP of about 13 years of age to about 17 years of age, or an adult having HPP of greater than about 18 years of age) can be assigned health state III if one or more of the following symptoms are present: slightly unusual appearance from tooth loss; shortened stature for their age; noticeably impaired mobility for their age; walk very slowly and awkwardly and may be clumsy; some muscle weakness and is not as strong or agile as healthy subject; problems completing their usual activities, such as dressing, going to school or work, and participating in sports; motor delay; can wash and dress themselves with some difficulties in ADL because of pain, muscle weakness, and other limitations in physical functioning; regularly experiences pain associated with their HPP; sleep may be disturbed due to pain or discomfort; health problems affect their self-esteem; mildly impaired psychological well-being and reduced social functioning; can attend school or work, but has problems completing tasks; some limitations to social life because of interrupted school/work life and the burden of living with HPP; occasionally experiences a lack of independence in their day to day life relative to healthy subject of same age; occasional respiratory problems; an increased risk of fractures and associated pain; premature loss of several teeth and other general dental complications; and a history of craniosynostosis and may have undergone skull surgery. The health state of the HPP patient can then be used as described herein to assign a treatment regimen to the patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa).

An HPP patient (e.g., a child having HPP of about 5 years of age to about 12 years of age, an adolescent having HPP of about 13 years of age to about 17 years of age, or an adult having HPP of greater than about 18 years of age) can be assigned health state IV if one or more of the following symptoms are present: noticeable skeletal deformities and an unusual appearance; shortened stature; struggles to walk to school or work and often relies upon a wheelchair or walking aids; significant muscle weakness and is significantly less agile than an otherwise healthy subject of their age; severe problems with any physical activity and could not participate in sport; gross motor delay; problems with washing and dressing; difficulties with their daily activities because of severe pain, muscle weakness, and other limitations in physical functioning; chronic pain that they receive medication for; sleep may be disturbed due to pain or discomfort; health problems have affected their self-esteem; diminished psychological wellbeing and fewer social contacts than a healthy subject of their age; school or work life is very disrupted by their HPP related problems and sometime have to spend days at home; unable to have a normal social life because of interrupted school/work life and the burden of living with HPP; a lack of independence in their day to day life relative to a healthy subject of the same age; prolonged respiratory infections; an increased risk of fractures and associated pain; premature loss of several teeth and other general dental complications; and a history of craniosynostosis and may have undergone skull surgery. The health state of the HPP patient can then be used as described herein to assign a treatment regimen to the patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa).

The aforementioned symptoms of HPP can be used singly or in combination with one or more metrics to classify the health status of an HPP patient. Exemplary metrics useful for assigning a treatment regimen based on a health state of an HPP patient or for assessing transition of an HPP patient from one health state to another are (1) the Six Minute Walk Test (6MWT), (2) EuroQol five dimension questionnaire (EQ-5D), (3) the Bruininks-Oseretsky Test of Motor Proficiency 2^{nd} Edition (BOT-2), (4) the Childhood Health Assessment Questionnaire (CHAQ), (5) the Pediatric Outcomes Data Collection Instrument (PODCI), which are described in further detail below. Another exemplary metric useful for assigning a treatment regimen based on a health state of an HPP patient or for assessing transition of an HPP patient from one health state to another is gait analysis, which is described, for example, in PCT Application No. PCT/US2016/039595, the disclosure of which is hereby incorporated by reference in its entirety.

### Six Minute Walk Test (6MWT)

A patient having HPP (e.g., a child having HPP of about 5 years of age to about 12 years of age, an adolescent having HPP of about 13 years of age to about 17 years of age, or an adult having HPP of greater than about 18 years of age) can be identified as having a health state (I, II, III, or IV) using the 6MWT. The health state of the HPP patient can then be used to assign a treatment regimen to the patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa). In particular, the 6MWT can be used to evaluate walking ability in a child having HPP of about 5 years of age to about 12 years of age, an adolescent having HPP of about 13 years of age to about 17 years of age, or an adult having HPP of greater than about 18 years of age to generate a 6MWT value for the child, adolescent, or adult, respectively.

The 6MWT can be performed indoors or outdoors using a flat, straight, enclosed corridor (e.g., of about 30 meters in length) with a hard surface. A stopwatch or other timer can be used to track the time and a mechanical counter or other device can be used to determine the distance (e.g., in meters) that the HPP patient (e.g., a child having HPP of about 5 years of age to about 12 years of age, an adolescent having HPP of about 13 years of age to about 17 years of age, or an adult having HPP of greater than about 18 years of age) walks. For instance, the length of the corridor can be marked every three meters to determine the number of meters walked by the HPP patient, with the turnaround point at 30 meters and the starting line also marked. The distance walked by the patient in six minutes can then be compared to the predicted number of meters walked, e.g., by an untreated subject of about the same age, the same gender, and/or the same height, and expressed as a percentage value to generate the 6MWT value of the patient. The 6MWT value of the patient (e.g., a child having HPP of about 5 years of age to about 12 years of age, an adolescent having HPP of about 13 years of age to about 17 years of age, or an adult having HPP of greater than about 18 years of age) can be compared to the 6MWT value at baseline of the patient. Additionally, the 6MWT value of the adult having HPP can be compared to the 6MWT value of a healthy patient.

A child having HPP of about 5 years of age to about 12 years of age can be identified as having health state IV when the patient has a 6MWT value of less than about 47.2% of a predicted 6MWT value for a healthy subject (e.g., the patient has a 6MWT value of about 10%, about 15%, about 20%, about 25%, about 30%, about 35%, about 40%, or about 45% of a predicted 6MWT value for a healthy subject of the same age, gender, and/or height); health state III when the patient has a 6MWT value of about 47.2% to about 64.8% of a predicted 6MWT value for a healthy subject (e.g., the patient has a 6MWT value of about 50%, about 52%, about 54%, about 56%, about 58%, about 60%, about 62% or about 64% of a predicted 6MWT value for a healthy subject of the same age, gender, and/or height); health state II when the patient has a 6MWT value of about 64.8% to about 82.4% of a predicted 6MWT value for a healthy subject (e.g., the patient has a 6MWT value of about 65%, about 67%, about 70%, about 73%, about 75%, about 77%, about 80% or about 82% of a predicted 6MWT value for a healthy subject of the same age, gender, and/or height); or health state I when the patient has a 6MWT value of greater than 82.4% of a predicted 6MWT value for a healthy subject (e.g., the patient has a 6MWT value of about 83%, about 85%, about 87%, about 90%, about 93%, about 95%, about 97% or about 100% of a predicted 6MWT value for a healthy subject of the same age, gender, and/or height).

The child having HPP of about 5 years of age to about 12 years of age can then be assigned a treatment regimen based on the health state of the child. For instance, an HPP child identified as having health state III or IV based on the 6MWT can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). An HPP child identified as having health state I or II based on the 6MWT can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

An adolescent having HPP of about 13 years of age to about 17 years of age can be identified as having health state IV when the patient has a 6MWT value of less than about 47.8% of a predicted 6MWT value for a healthy subject (e.g., the patient has a 6MWT value of about 10%, about 15%, about 20%, about 25%, about 30%, about 35%, about 40%, about 45%, or about 47% of a predicted 6MWT value for a healthy subject of the same age, gender, and/or height); health state III when the patient has a 6MWT value of about 47.8% to about 65.2% of a predicted 6MWT value for a healthy subject (e.g., the patient has a 6MWT value of about 50%, about 52%, about 54%, about 56%, about 58%, about 60%, about 62%, about 64%, or about 65% of a predicted 6MWT value for a healthy subject of the same age, gender, and/or height); health state II when the patient has a 6MWT value of about 65.2% to about 82.6% of a predicted 6MWT value for a healthy subject (e.g., the patient has a 6MWT value of about 66%, about 67%, about 70%, about 73%, about 75%, about 77%, about 80% or about 82% of a predicted 6MWT value for a healthy subject of the same age, gender, and/or height); or health state I when the patient has a 6MWT value of greater than 82.6% of a predicted 6MWT value for a healthy subject (e.g., the patient has a 6MWT value of about 83%, about 85%, about 87%, about 90%, about 93%, about 95%, about 97%, or about 100% of a predicted 6MWT value for a healthy subject of the same age, gender, and/or height).

The adolescent having HPP of about 13 years of age to about 17 years of age can then be assigned a treatment regimen based on the health state of the adolescent. For instance, an HPP adolescent identified as having health state III or IV based on the 6MWT can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). An HPP adolescent identified as having health state I or II based on the 6MWT can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

An adult having HPP of greater than about 18 years of age or older can be identified as having health state IV when the patient has a 6MWT value of less than about 52.0% of a predicted 6MWT value for a healthy subject (e.g., the patient has a 6MWT value of about 10%, about 15%, about 20%, about 25%, about 30%, about 35%, about 40%, about 45%, about 47%, about 49%, or about 51% of a predicted 6MWT value for a healthy subject of the same age, gender, and/or height); health state III when the patient has a 6MWT value of about 52.0% to about 68.0% of a predicted 6MWT value for a healthy subject (e.g., the patient has a 6MWT value of about 53%, about 54%, about 56%, about 58%, about 60%, about 62%, about 64%, about 65%, about 66%, or about 67% of a predicted 6MWT value for a healthy subject of the same age, gender, and/or height); health state II when the patient has a 6MWT value of about 68.0% to about 84.0% of a predicted 6MWT value for a healthy subject (e.g., the patient has a 6MWT value of about 69%, about 70%, about 73%, about 75%, about 77%, about 80%, about 82%, or about 83% of a predicted 6MWT value for a healthy subject of the same age, gender, and/or height); or health state I when the patient has a 6MWT value of greater than 84.0% of a predicted 6MWT value for a healthy subject (e.g., the patient has a 6MWT value of about 85%, about 87%, about 90%, about 93%, about 95%, about 97%, or about 100% of a predicted 6MWT value for a healthy subject of the same age, gender, and/or height).

The adult having HPP of about 18 years of age or older can then be assigned a treatment regimen based on the health state of the adult. For instance, an HPP adult identified as having health state III or IV based on the 6MWT can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). An HPP adult identified as having health state I or II based on the 6MWT can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

The methods can result in an improvement in the 6MWT value of an HPP patient (e.g., a child having HPP of about 5 years of age to about 12 years of age, an adolescent having HPP of about 13 years of age to about 17 years of age, or an adult having HPP of greater than about 18 years of age or older). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks), can result in an increase in the 6MWT value of the patient and transition of the patient to an improved health status (e.g., from a health state of IV to III, IV to II, IV to I, III to II, III to I, or II to I). For example, assignment of a treatment regimen based on a health state of an HPP child that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the child; particularly at least six weeks, e.g., at least 96 weeks), can result in an increase in the 6MWT value of the child from less than about 47.2% to 47.2% to about 64.8% (a health state of III), from below 64.8% to about 64.8% to about 82.4% (a health state of II), or from below 82.4% to above 82.4% (a health state of I). Likewise, assignment of a treatment regimen based on a health state of an HPP adolescent that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the adolescent; particularly at least six weeks, e.g., at least 96 weeks), can result in an increase in the 6MWT value of the adolescent from less than about 47.8% to 47.8% to about 65.2% (a health state of III), from below 65.2% to about 65.2% to about 82.6% (a health state of II), or from below 82.6% to above 82.6% (a health state of I). Additionally, assignment of a treatment regimen based on a health state of an HPP adult that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the adult; particularly at least six weeks, e.g., at least 96 weeks), can result in an increase in the 6MWT value of the adult from less than about 52.0% to 52.0% to about 68.0% (a health state of III), from below 68.0% to about 68.0% to about 84.0% (a health state of II), or from below 84.0% to above 84.0% (a health state of I).

The increase in the 6MWT value of the HPP patient (e.g., a child having HPP of about 5 years of age to about 12 years of age, an adolescent having HPP of about 13 years of age to about 17 years of age, or an adult having HPP of greater than about 18 years of age or older) can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). For instance, the 6MWT value increases to greater than about 80% of the predicted 6MWT value of the patient and remains at ±10% of the increased 6MWT value during treatment with the sALP (e.g., asfotase alfa).

Alternatively, when administration of an sALP does not result in an increase in the 6MWT value of a HPP patient and a transition to an improved health status, the dosage and/or frequency of sALP administration can be changed in order to determine the effective amount of the sALP for the HPP patient (e.g., a child having HPP of about 5 years of age to about 12 years of age, an adolescent having HPP of about 13 years of age to about 17 years of age, or an adult having HPP of greater than about 18 years of age or older). For instance, the dosage of the sALP (such as TNALP, for example the sALP fusion polypeptide of SEQ ID NO: 1 or a polypeptide variant having at least 95% sequence identity to the sequence of SEQ ID NO: 1, e.g., asfotase alfa) can be increased from, e.g., about 2.1 mg/kg/week or about 3.5 mg/kg/week to about 6 mg/kg/week or about 9 mg/kg/week, when the patient exhibits a decrease of one or more levels, or does not exhibit an improvement of at least one level, in the health status.

### EuroQol Five Dimension Questionnaire (EQ-5D)

A patient having HPP (e.g., a child having HPP of about 5 years of age to about 12 years of age, an adolescent having HPP of about 13 years of age to about 17 years of age, or an adult having HPP of greater than about 18 years of age) can be identified as having a health state (I, II, III, or IV) using the EuroQol five dimension questionnaire (EQ-5D), e.g., in combination with one or more of the other metrics described herein, such as the 6MWT. The health state of the HPP patient can then be used to assign a treatment regimen to the HPP patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa). In particular, the EQ-5D can be used to evaluate walking ability in a child having HPP of about 5 years of age to about 12 years of age, an adolescent having HPP of about 13 years of age to about 17 years of age, or an adult having HPP of greater than about 18 years of age or older to generate a 6MWT value for the child, adolescent, or adult, respectively.

The EQ-5D used to assess the health state of a HPP patient includes, e.g., the dimensions of mobility, self-care, ability to perform ADLs (e.g., work, study, housework, family, or leisure activities), incidence of pain or discomfort, and anxiety or depression. For a description of the EQ-5D index, see Reenan & Oppe (EQ-5D-3L User Guide Version 5.1, 2015), hereby incorporated by reference in its entirety. The EQ-5D may be administered by a clinician or in an interview. After completing the EQ-5D, the HPP patient is then categorized as having a health state of level I indicating no problems with physiological condition, level II indicating some problems with physiological condition, level III indicating extreme problems with physiological condition, or level IV indicating the most extreme problems of physiological condition. The EQ-5D can also be used to assess the transition of an HPP patient from one health state to another health state, such as from a health state of IV to III, IV to II, IV to I, III to II, III to I, or II to I. In particular, the EQ-5D is performed in combination with the 6MWT to assess the health state level of patient or a transition from one health state to another.

The health state of a patient having HPP (e.g., a child having HPP of about 5 years of age to about 12 years of age, an adolescent having HPP of about 13 years of age to about 17 years of age, or an adult having HPP of greater than about 18 years of age or older) is determined, e.g., using the 6MWT as the physical assessment and is further identified as having health state IV when the patient has an EQ-5D value of less than about 0.23 (e.g., about 0.1, 0.12, 0.14, 0.16, 0.18, 0.20, or 0.22); health state III when the patient has an EQ-5D value of about 0.23 to about 0.54 (e.g., about 0.25, 0.30, 0.35, 0.40, 0.50, or 0.52), health state II when the patient has an EQ-5D value of about 0.54 to about 0.67 (e.g., about 0.55, 0.57, 0.60, 0.63, 0.65, or 0.66), and health state I when the patient has an EQ-5D value of greater than about 0.67 (e.g., about 0.68, 0.70, 0.75, 0.80, 0.85, or 0.90 or greater). The patient having HPP can then be assigned a treatment regimen based on the health state of the patient. For instance, a patient identified as having health state III or IV based on the EQ-5D can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). A patient identified as having health state I or II based on the EQ-5D can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

The methods can result in an improvement in the EQ-5D of an HPP patient (e.g., a child having HPP of about 5 years of age to about 12 years of age, an adolescent having HPP of about 13 years of age to about 17 years of age, or an adult having HPP of greater than about 18 years of age or older). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks), can result in an increase in the EQ-5D of the patient and transition of the patient to an improved health status (e.g., from a health state of IV to III, IV to II, IV to I, III to II, III to I, or II to I).

For example, assignment of a treatment regimen based on a health state of a patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the child; particularly at least six weeks, e.g., at least 96 weeks), can result in an increase in the EQ-5D of the child from less than about 0.23 to about 0.23 to about 0.54 (a health state of III), from below 0.54 to about 0.54 to about 0.67 (a health state of II), or from below 0.67 to above 0.67 (a health state of I).

The increase in the EQ-5D of the HPP patient (e.g., a child having HPP of about 5 years of age to about 12 years of age, an adolescent having HPP of about 13 years of age to about 17 years of age, or an adult having HPP of greater than about 18 years of age or older) can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). For instance, the EQ-5D increases to greater than about 0.67 and remains at ±10% of the increased EQ-5D value during treatment with the sALP (e.g., asfotase alfa).

Alternatively, when administration of an sALP does not result in an increase in the EQ-5D value of a HPP patient and a transition to an improved health status, the dosage and/or frequency of sALP administration can be changed in order to determine the effective amount of the sALP for the HPP patient (e.g., a child having HPP of about 5 years of age to about 12 years of age, an adolescent having HPP of about 13 years of age to about 17 years of age, or an adult having HPP of greater than about 18 years of age or older). For instance, the dosage of the sALP (such as TNALP, for example the sALP fusion polypeptide of SEQ ID NO: 1 or a polypeptide variant having at least 95% sequence identity to the sequence of SEQ ID NO: 1, e.g., asfotase alfa) can be increased from, e.g., about 2.1 mg/kg/week or about 3.5 mg/kg/week to about 6 mg/kg/week or about 9 mg/kg/week, when the patient exhibits a decrease of one or more levels, or does not exhibit an improvement of at least one level, in the health status.

### Bruininks-Oseretsky Test of Motor Proficiency 2^{nd} Edition (BOT-2)

Patients having HPP (e.g., a child having HPP of about 5 years of age to about 12 years of age, an adolescent having HPP of about 13 years of age to about 17 years of age, or an adult having HPP of greater than about 18 years of age or older) can be can be identified as having a health state (I, II, III, or IV) using the Bruininks-Oseretsky Test of Motor Proficiency 2nd Edition (BOT-2) running speed and agility and BOT-2 strength tests. The health state of the HPP patient can then be used to assign a treatment regimen to the HPP patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa). In particular, the BOT-2 speed and agility and BOT-2 strength tests can be used to evaluate physical impairments and mobility restrictions in an adult having HPP to generate a total BOT-2 speed and agility score and/or total BOT-2 strength score for the adult.

The BOT-2 includes a range of tests to evaluate physical impairments of a patient having HPP (e.g., a child having HPP of about 5 years of age to about 12 years of age, an adolescent having HPP of about 13 years of age to about 17 years of age, or an adult having HPP of greater than about 18 years of age or older), which can be performed with, e.g., a kit including the tests. The BOT-2 provides composite BOT-2 scores in the following areas: strength, running speed and agility, fine motor precision, fine motor integration, manual dexterity, bilateral coordination, balance, and upper-limb coordination. For example, the patient having HPP can perform sit-ups, v-ups, standing long jump, wall sit, and/or push-ups to determine the BOT-2 strength score; the patient having HPP can step over a balance beam and/or perform a shuttle run, two-legged side hop, and/or one-legged side hop to determine the BOT-2 running speed and agility score; the patient having HPP can cut out a circle and/or connect dots to determine the BOT-2 fine motor precision score; the patient having HPP can copy a star and/or copy a square to determine the BOT-2 fine motor integration score; the patient having HPP can transfer pennies, sort cards, and/or string blocks to determine the manual dexterity score; the patient having HPP can tap his or her foot and finger and/or perform jumping jacks to determine the BOT-2 bilateral coordination score; the patient having HPP can walk forward on a line and/or stand on one leg on a balance beam to determine the BOT-2 balance score; and the patient having HPP can throw a ball at a target and/or catch a tossed ball to determine the BOT-2 upper-limb coordination score. The BOT-2 score is an additive total of each area assessed. Moreover, the BOT-2 score used to assess the physical proficiency of the patient can be the raw additive score or a normative score.

A patient having HPP (e.g., a child having HPP of about 5 years of age to about 12 years of age, an adolescent having HPP of about 13 years of age to about 17 years of age, or an adult having HPP of greater than about 18 years of age or older) could perform tests in one or more of described areas (strength, running speed and agility, fine motor precision, fine motor integration, manual dexterity, bilateral coordination, balance, and upper-limb coordination) to generate a BOT-2 score indicative of physical impairments in the patient. Within each BOT-2 area (strength, running speed and agility, fine motor precision, fine motor integration, manual dexterity, bilateral coordination, balance, and upper-limb coordination), a patient having HPP could perform one or more tests to determine the BOT-2 score of the patient, e.g., the patient could perform one or more of sit-ups, v-ups, standing long jump, wall sit, and push-ups to determine the BOT-2 strength score. If desired, only a single test (e.g., one test selected from the group of sit-ups, v-ups, standing long jump, wall sit, and push-ups) can be performed to determine the BOT-2 score (e.g., a BOT-2 strength score) of patient having HPP.

Each of the BOT-2 scores (strength, running speed and agility, fine motor precision, fine motor integration, manual dexterity, bilateral coordination, balance, and upper-limb coordination) of the patient having HPP (e.g., a child having HPP of about 5 years of age to about 12 years of age, an adolescent having HPP of about 13 years of age to about 17 years of age, or an adult having HPP of greater than about 18 years of age or older) can be compared to the BOT-2 score of patients without HPP (e.g., a child without HPP of about 5 years of age to about 12 years of age, an adolescent without HPP of about 13 years of age to about 17 years of age, or an adult without HPP of greater than about 18 years of age or older, respectively) to, e.g., determine the standard deviation of the BOT-2 score. Each of the BOT-2 scores (e.g., strength, running speed and agility, fine motor precision, fine motor integration, manual dexterity, bilateral coordination, balance, and upper-limb coordination) of the patient having HPP can be compared to the BOT-2 score of other HPP patients (e.g., HPP patients of about the same age, height, and/or gender) to, e.g., determine the BOT-2 score for the HPP patient.

BOT-2 scores (e.g., strength, running speed and agility, fine motor precision, fine motor integration, manual dexterity, bilateral coordination, balance, and upper-limb coordination scores) range from about 0 to equal to or less than about 25, in which a score of greater than about 10 is considered representative of healthy subjects (e.g., patients without HPP). Patients with a BOT-2 score (e.g., strength, running speed and agility, fine motor precision, fine motor integration, manual dexterity, bilateral coordination, balance, and upper-limb coordination scores) of less than about 10 can be treated with an sALP (such as TNALP, for example the sALP fusion polypeptide of SEQ ID NO: 1 or a polypeptide variant having at least 95% sequence identity to the sequence of SEQ ID NO: 1, e.g., asfotase alfa), such as by administering an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

For example, a patient having HPP (e.g., a child having HPP of about 5 years of age to about 12 years of age, an adolescent having HPP of about 13 years of age to about 17 years of age, or an adult having HPP of greater than about 18 years of age) is identified as having health state IV when the patient has a BOT-2 running speed and agility score or BOT-2 strength score of less than 3 (e.g., about 0.5, 1, 1.5, 2, 2.5, or 3); health state III when the patient has a BOT-2 running speed and agility score or BOT-2 strength score of about 3 to about 7 (e.g., about 3.5, 4.0, 4.5, 5.0,5.5, 6.0, or 6.5), health state II when the patient has a BOT-2 running speed and agility score or BOT-2 strength score of about 7 to about 10 (e.g., about 7.5, 8.0, 8.5, 9.0, or 9.5), and health state I when the patient has a BOT-2 running speed and agility score or BOT-2 strength score of greater than 10 (e.g., 11, 12, 13, 14, 15, 16, 17, 18, 19, 20, 21, 22, 23, 24, or 25). The patient having HPP can then be assigned a treatment regimen based on the health state of the patient as identified using BOT-2 running speed and agility score and/or BOT-2 strength score.

For instance, an HPP patient (e.g., a child having HPP of about 5 years of age to about 12 years of age, an adolescent having HPP of about 13 years of age to about 17 years of age, or an adult having HPP of greater than about 18 years of age or older) identified as having health state III or IV based on the BOT-2 running speed and agility score or BOT-2 strength score can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). A patient identified as having health state I or II based on the BOT-2 running speed and agility score or BOT-2 strength score can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

The methods can result in an improvement in the BOT-2 score (e.g., strength, running speed and agility, fine motor precision, fine motor integration, manual dexterity, bilateral coordination, balance, and/or upper-limb coordination score) of an HPP patient (e.g., a child having HPP of about 5 years of age to about 12 years of age, an adolescent having HPP of about 13 years of age to about 17 years of age, or an adult having HPP of greater than about 18 years of age or older). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks), can result in an increase in the BOT-2 running speed and agility score from below 3 to about 3 to about 7 (a health state of III), from below 7 to about 7 to about 10 (a health state of II), or from below 10 to above 10 (a health state of I). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks), can result in an increase in the BOT-2 strength score from below 3 to about 3 to about 7 (a health state of III), from below 7 to about 7 to about 10 (a health state of II), or from below 10 to above 10 (a health state of I).

The increase in the BOT-2 score (e.g., strength, running speed and agility, fine motor precision, fine motor integration, manual dexterity, bilateral coordination, balance, and/or upper-limb coordination score) of an HPP patient (e.g., a child having HPP of about 5 years of age to about 12 years of age, an adolescent having HPP of about 13 years of age to about 17 years of age, or an adult having HPP of greater than about 18 years of age or older) can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). Likewise, the improvement in the health state of the patient can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

Additionally, within each BOT-2 area (strength, running speed and agility, fine motor precision, fine motor integration, manual dexterity, bilateral coordination, balance, and upper-limb coordination), an HPP patient (e.g., a child having HPP of about 5 years of age to about 12 years of age, an adolescent having HPP of about 13 years of age to about 17 years of age, or an adult having HPP of greater than about 18 years of age) could perform one or more tests to determine the BOT-2 score of the patient and assign a treatment regimen based on the health state of an HPP patient as identified using the BOT-2 score. For instance, the HPP patient could perform one or more of sit-ups, V-ups, standing long jump, wall sit, and push-ups to determine the BOT-2 strength score and assess the treatment efficacy of sALP administration. The HPP patient could perform one or more of balance beam, a shuttle run, two-legged side hop, and/or one-legged side hop to determine the BOT-2 running speed and agility score and assess the treatment efficacy of sALP administration. The HPP patient can cut out a circle and/or connect dots to determine the BOT-2 fine motor precision score and assess the treatment efficacy of sALP administration. The HPP patient can copy a star and/or copy a square to determine the BOT-2 fine motor integration score and assess the treatment efficacy of sALP administration. The HPP patient could perform one or more of transferring pennies, sorting cards, and stringing blocks to determine the BOT-2 manual dexterity score and assess the treatment efficacy of sALP administration. The HPP patient can tap his or her foot and finger and/or perform jumping jacks to determine the BOT-2 bilateral coordination score and assess the treatment efficacy of sALP administration. The HPP patient can walk forward on a line and/or stand on one leg on a balance beam to determine the BOT-2 balance score and assess the treatment efficacy of sALP administration. The HPP patient can throw a ball at a target and/or catch a tossed ball to determine the BOT-2 upper-limb coordination score and assess the treatment efficacy of sALP administration.

Alternatively, when assignment of a treatment regimen including administration of an sALP does not result in an increase in the BOT-2 running speed and agility score to greater than about 10 (e.g., an increase of at least 2 to 10 points (e.g., 2, 3, 4, 5, 6, 7, 8, 9, or 10 points) over the BOT-2 running speed and agility score prior to treatment with the sALP) and an improvement to a health state of I, the dosage and/or frequency of sALP administration can be changed in order to determine the effective amount of the sALP for the HPP patient (e.g., a child having HPP of about 5 years of age to about 12 years of age, an adolescent having HPP of about 13 years of age to about 17 years of age, or an adult having HPP of greater than about 18 years of age or older). For instance, the dosage of the sALP (such as TNALP, for example the sALP fusion polypeptide of SEQ ID NO: 1 or a polypeptide variant having at least 95% sequence identity to the sequence of SEQ ID NO: 1, e.g., asfotase alfa) can be increased from, e.g., about 2.1 mg/kg/week or about 3.5 mg/kg/week to about 6 mg/kg/week or about 9 mg/kg/week.

### Childhood Health Assessment Questionnaire (CHAQ)

The Childhood Health Assessment Questionnaire (CHAQ) can be administered to identify the health state of a patient having HPP (e.g., a child having HPP of about 5 years of age to about 12 years of age or an adolescent having HPP of about 13 years of age to about 17 years of age) to generate a CHAQ index score for the child, as is described in Bruce & Fries (J. Rheumatol. 30(1): 167-178, 2003) and Klepper (Arthritis & Rheumatism, 49: S5-S14, 2003), hereby incorporated by reference in their entirety. The health state of the HPP patient can then be used to assign a treatment regimen to the HPP patient including administration of an sALP (such as TNALP, for example the sALP fusion polypeptide of SEQ ID NO: 1 or a polypeptide variant having at least 95% sequence identity to the sequence of SEQ ID NO: 1, e.g., asfotase alfa). The CHAQ includes eight categories of questions for dressing/grooming, arising, eating, walking, hygiene, reach, grip, and activities, in which a parent or guardian records the amount of difficulty the patient with the muscle weakness disease (e.g., HPP) has in performing the respective activities. The range of scores within each category is from 0 to 3, in which a score of 0 indicates without any difficulty; a score of 1 indicates with some difficulty; a score of 2 indicates with much difficulty; and a score of 3 indicates that the patient is unable to perform the activity.

For example, a patient having HPP (e.g., a child having HPP of about 5 years of age to about 12 years of age or an adolescent having HPP of about 13 years of age to about 17 years of age) is identified as having health state IV when the patient has a CHAQ index score of about 2.5 to about 3 (e.g., about 2.5, 2.6, 2.7, 2.8, 2.9, or 3.0); health state III when the patient has a CHAQ index score of about 2.0 to about 2.5 (e.g., about 2.1, 2.2, 2.3, 2.4, or 2.5); health state II when the patient has a CHAQ index score of about 1.0 to about 2.0 (e.g., about 1.1, 1.2, 1.3, 1.4, 1.5, 1.6, 1.7, 1.8, 1.9, or 2.0); or health state I when the patient has a CHAQ index score of less than about 1 (e.g., about 0.1, 0.2, 0.3, 0.4, 0.5, 0.6, 0.7, 0.8, or 0.9). The patient having HPP can then be assigned a treatment regimen based on the health state of the patient as identified using a CHAQ index score.

For instance, an HPP patient (e.g., a child having HPP of about 5 years of age to about 12 years of age or an adolescent having HPP of about 13 years of age to about 17 years of age) identified as having health state III or IV based on the CHAQ index score can be assigned a treatment regimen including administering, e.g., about 6 mg/kg/week to about 9 mg/kg/week, preferably 6 mg/kg/week, of an sALP (such as TNALP, for example the sALP fusion polypeptide of SEQ ID NO: 1 or a polypeptide variant having at least 95% sequence identity to the sequence of SEQ ID NO: 1, e.g., asfotase alfa) to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). A patient identified as having health state I or II based on the CHAQ index score can be assigned a treatment regimen including administering, e.g., about 1 mg/kg/week to about 6 mg/kg/week, preferably 6 mg/kg/week, of the sALP to the patient for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

The methods can result in an improvement in the CHAQ index score of an HPP patient (e.g., a child having HPP of about 5 years of age to about 12 years of age or an adolescent having HPP of about 13 years of age to about 17 years of age). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks), can result in a decrease in the CHAQ index score from above 2.5 to about 2.0 to about 2.5 (a health state of III), from above 2.0 to about 1.0 to about 2.0 (a health state of II), or from above 1.0 to below 1.0 (a health state of I).

The decrease in the CHAQ index score of an HPP patient (e.g., a child having HPP of about 5 years of age to about 12 years of age or an adolescent having HPP of about 13 years of age to about 17 years of age) can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). Likewise, the improvement in the health state of the patient can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

Alternatively, when assignment of a treatment regimen including administration of an sALP does not result in a decrease in the CHAQ index score or and an improvement in a health state of the HPP patient (e.g., a child having HPP of about 5 years of age to about 12 years of age or an adolescent having HPP of about 13 years of age to about 17 years of age), the dosage and/or frequency of sALP administration can be changed in order to determine the effective amount of the sALP for the HPP patient (e.g., a child having HPP of about 5 years of age to about 12 years of age or an adolescent having HPP of about 13 years of age to about 17 years of age). For instance, the dosage of the sALP (such as TNALP, for example the sALP fusion polypeptide of SEQ ID NO: 1 or a polypeptide variant having at least 95% sequence identity to the sequence of SEQ ID NO: 1, e.g., asfotase alfa) can be increased from, e.g., about 2.1 mg/kg/week or about 3.5 mg/kg/week to about 6 mg/kg/week or about 9 mg/kg/week, if the HPP patient does not exhibit a decrease in CHAQ index score and an improvement of at least one level in health status after treatment with the sALP.

### Pediatric Outcomes Data Collection Instrument (PODCI)

An HPP patient (e.g., a child having HPP of about 5 years of age to about 12 years of age or an adolescent having HPP of about 13 years of age to about 17 years of age) can be identified for treatment with an sALP (e.g., TNALP, for example the sALP polypeptide of SEQ ID NO: 1 or a polypeptide variant having at least 95% sequence identity to the sequence of SEQ ID NO: 1, e.g., asfotase alfa) using the Pediatric Outcomes Data Collection Instrument (PODCI). The PODCI can be administered to evaluate the health status of patients to generate a PODCI score for the patient, as is described in Plint et al. (J. Pediatr. Orthop. 23(6): 788-790, 2003). The PODCI includes eight categories of questions that can be completed by an HPP patient or by a parent/guardian of the subject. Categories that can be used to determine the PODCI of an HPP patient include the following: 1) the upper extremity and physical function scale to measure difficulty encountered in performing daily personal care and student activities; 2) the transfer and basic mobility scale to measure difficulty experienced in performing routine motion and motor activities in daily activities; 3) the sports/physical functioning scale to measure difficulty or limitations encountered in participating in more active activities or sports; 4) the pain/comfort scale to measure the level of pain experienced during the past week; 5) the treatment expectations scale to measure the long term expectations of treatment; 6) the happiness scale to measure overall satisfaction with personal looks and sense of similarity to friends and others of own age; 7) the satisfaction with symptoms scale to measure the patient's acceptance of current limitations should this be a life-long state; and 8) the global functioning scale, which is a general combined scale calculated from the first four scales listed above. In each of the categories, a standardized score is determined for the HPP patient and then converted to a 0 to 100 scale, in which 0 represents significant disability and 100 represents less disability.

For example, a patient having HPP (e.g., a child having HPP of about 5 years of age to about 12 years of age or an adolescent having HPP of about 13 years of age to about 17 years of age) is identified as having health state IV when the patient has a PODCI score (e.g., indicative of disability in ADL and/or pain) of less than 25 (e.g., about 5, about 10, about 15, about 20, or about 25); health state III when the patient has a PODCI score of about 25 to about 50 (e.g., about 25, about 30, about 35, about 40, about 45, or about 50), health state II when the patient has a PODCI index score of about 50 to about 75 (e.g., about 55, about 60, about 65, about 70, or about 75); or health state I when the patient has a PODCI index score of greater than 75 (e.g., about 80, about 85, about 90, about 95, or about 100). The patient having HPP can then be assigned a treatment regimen based on the health state of the patient as identified using a PODCI score.

The methods can result in an improvement in the PODCI score of an HPP patient (e.g., a child having HPP of about 5 years of age to about 12 years of age or an adolescent having HPP of about 13 years of age to about 17 years of age). For example, assignment of a treatment regimen based on a health state of an HPP patient that includes administering an sALP, such as treatment with an sALP for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks), can result in an increase in the PODCI score from less than 25 to about 25 to about 50 (a health state of III), from less than 50 to about 50 to about 75 (a health state of II), or less than 75 to about 75 to about 100 (a health state of I).

The increase in the PODCI score of an HPP patient (e.g., a child having HPP of about 5 years of age to about 12 years of age or an adolescent having HPP of about 13 years of age to about 17 years of age) can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks). Likewise, the improvement in the health state of the patient can be sustained throughout administration of the sALP (such as TNALP, for example the sALP fusion polypeptide of SEQ ID NO: 1 or a polypeptide variant having at least 95% sequence identity to the sequence of SEQ ID NO: 1, e.g., asfotase alfa), e.g., for a treatment period of at least two weeks (e.g., at least three weeks, at least four weeks, at least five weeks, at least six weeks, at least seven weeks, at least eight weeks, at least nine weeks, at least ten weeks, at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six weeks, e.g., at least 96 weeks).

Alternatively, when assignment of a treatment regimen including administration of an sALP does not result in an increase in the PODCI score or and an improvement in a health state of the HPP patient (e.g., a child having HPP of about 5 years of age to about 12 years of age or an adolescent having HPP of about 13 years of age to about 17 years of age), the dosage and/or frequency of sALP administration can be changed in order to determine the effective amount of the sALP for the HPP patient (e.g., a child having HPP of about 5 years of age to about 12 years of age or an adolescent having HPP of about 13 years of age to about 17 years of age). For instance, the dosage of the sALP (such as TNALP, for example the sALP fusion polypeptide of SEQ ID NO: 1 or a polypeptide variant having at least 95% sequence identity to the sequence of SEQ ID NO: 1, e.g., asfotase alfa) can be increased from, e.g., about 2.1 mg/kg/week or about 3.5 mg/kg/week to about 6 mg/kg/week or about 9 mg/kg/week, if the HPP patient does not exhibit an increase in PODCI score and an improvement of at least one level in health status after treatment with the sALP.

### Alkaline Phosphatase

Asfotase alfa is a human TNALP (hTNALP; SEQ ID NO: 1) fusion polypeptide formulated for the treatment of hypophosphatasia (HPP). In particular, asfotase alfa (SEQ ID NO: 1) can be used effectively to treat HPP, its symptoms, and physical impairments associated therewith in patients having HPP (e.g., a child having HPP of about 5 years of age to about 12 years of age, an adolescent having HPP of about 13 years of age to about 17 years of age, or an adult having HPP of greater than about 18 years of age or older), particularly HPP patients identified as having a health state of IV, III, II, or I.

Given the results described herein, the methods are not limited to administration of a particular alkaline phosphatase (ALP) or nucleic acid sequence encoding an ALP. Alkaline phosphatases encompass a group of enzymes that catalyze the cleavage of a phosphate moiety (e.g., hydrolysis of pyrophosphate, PPᵢ). There are four known mammalian alkaline phosphatase (ALP) isozymes: tissue nonspecific alkaline phosphatase (TNALP; described further below), placental alkaline phosphatase (PLALP) (e.g., Accession Nos. P05187, NP_112603, and NP_001623), germ cell alkaline phosphatase (GALP) (e.g., Accession No. P10696), and intestinal alkaline phosphatase (IALP) (e.g., Accession Nos. P09923 and NP_001622). In addition to the exemplary ALPs discussed above, any polypeptide having the identical or similar catalytic site structure and/or enzymatic activity of ALP can be used (e.g., as an sALP or an sALP fusion polypeptide as defined herein) for treating an HPP patient, such as a child having HPP of about 5 years of age to about 12 years of age, an adolescent having HPP of about 13 years of age to about 17 years of age, or an adult having HPP of greater than about 18 years of age. Bone delivery conjugates including sALP are further described in PCT publication Nos: WO 2005/103263 and WO 2008/138131.

TNALPs that can be used according to the methods described herein include, e.g., human TNALP (Accession Nos. NP_000469, AAI10910, AAH90861, AAH66116, AAH21289, and AA126166); rhesus TNALP (Accession No. XP_01109717); rat TNALP (Accession No. NP_037191); dog TNALP (Accession No. AAF64516); pig TNALP (Accession No. AAN64273), mouse (Accession No. NP_031457), cow TNALP (Accession Nos. NP_789828, NP_776412, AAM 8209, and AAC33858), and cat TNALP (Accession No. NP_001036028). In particular, TNALP can be a recombinant human TNALP (e.g., SEQ ID NO: 1, asfotase alfa; see U.S. Pat. Nos. 7,763,712 and 7,960,529, incorporated herein by reference in their entirety) used for the treatment of an HPP patient, such as a child having HPP of about 5 years of age to about 12 years of age, an adolescent having HPP of about 13 years of age to about 17 years of age, or an adult having HPP of greater than about 18 years of age. The TNALP can also be one that exhibits at least about 95% sequence identity to the polypeptide or nucleic acid sequence of the above-noted TNALPs.

### Soluble Alkaline Phosphatases

The ALPs that can be used in the methods described herein include soluble (e.g., extracellular or non-membrane-bound) forms of any of the alkaline phosphatases described herein. The sALP can be, for example, a soluble form of human tissue non-specific alkaline phosphatase (human TNALP (hTNALP)). The methods are not limited to a particular sALP and can include any sALP that is physiologically active toward, e.g., phosphoethanolamine (PEA), inorganic pyrophosphate (PPi), and pyridoxal 5′-phosphate (PLP). In particular, an sALP is one that is catalytically competent to improve skeletal mineralization in bone. The methods further include nucleic acids encoding the sALPs described herein that can be used to treat the conditions described herein, e.g., HPP, such as patient with HPP (e.g., a child having HPP of about 5 years of age to about 12 years of age, an adolescent having HPP of about 13 years of age to about 17 years of age, or an adult having HPP of greater than about 18 years of age or older).

TNALP is a membrane-bound protein anchored by a glycolipid moiety at the C-terminal (Swiss-Prot, P05186). This glycolipid anchor (GPI) is added post-translationally after the removal of a hydrophobic C-terminal end, which serves both as a temporary membrane anchor and as a signal for the addition of the GPI. While the GPI anchor is located in the cell membrane, the remaining portions of TNALP are extracellular. In particular, TNALP (e.g., human TNALP (hTNALP)) can be engineered to replace the first amino acid of the hydrophobic C-terminal sequence (an alanine) with a stop codon, thereby producing an engineered hTNALP that contains all amino acid residues of the native anchored form of TNALP and lacks the GPI membrane anchor. One skilled in the art will appreciate that the position of the GPI membrane anchor will vary in different ALPs and can include, e.g., the last 10, 12, 14, 16, 18, 20, 21, 22, 23, 24, 25, 26, 27, 28, 29, 30, 32, 34, 36, 38, 40, 45, 50, or more amino acid residues on the C-terminus of the polypeptide. Recombinant sTNALP can include, e.g., amino acids 1 to 502 (18 to 502 when secreted), amino acids 1 to 501 (18 to 501 when secreted), amino acids 1 to 504 (18 to 504 when secreted), amino acids 1 to 505 (18-505 when secreted), or amino acids 1 to 502 of, e.g., SEQ ID NOs: 2-6. Thus, the C-terminal end of the native ALP can be truncated by certain amino acids without affecting ALP activity.

In addition to the C-terminal GPI anchor, TNALP also has an N-terminal signal peptide sequence. The N-terminal signal peptide is present on the synthesized protein when it is synthesized, but cleaved from TNALP after translocation into the ER. The sALPs include both secreted (i.e., lacking the N-terminal signal) and non-secreted (i.e., having the N-terminal signal) forms thereof. One skilled in the art will appreciate that the position of the N-terminal signal peptide will vary in different alkaline phosphatases and can include, for example, the first 5, 8, 10, 11, 12, 13, 14, 15, 16, 17, 18, 19, 20, 21, 22, 23, 24, 25, 27, 30, or more amino acid residues on the N-terminus of the polypeptide. One of skill in the art can predict the position of a signal sequence cleavage site, e.g., by an appropriate computer algorithm such as that described in Bendtsen et al. (J. Mol. Biol. 340(4):783-795, 2004) and available on the Web at www.cbs.dtu.dk/services/SignalP/.

The methods can also be performed using sALP consensus sequences derived from the extracellular domain of ALP isozymes (e.g., TNALP, PALP, GCALP, IALP, etc.). Thus, similar to sTNALP discussed above, the present disclosure also provides other soluble human ALP isozymes, i.e., without the peptide signal, preferably comprising the extracellular domain of the ALPs. The sALPs also include polypeptide sequences satisfying a consensus sequence derived from the ALP extracellular domain of human ALP isozymes and of mammalian TNALP orthologs (human, mouse, rat, cow, cat, and dog) or a consensus derived from the ALP extracellular domain of just mammalian TNALP orthologs (human, mouse, rat, cow, cat, and dog). The sALPs also include those which satisfy similar consensus sequences derived from various combinations of these TNALP orthologs or human ALP isozymes. Such consensus sequences are given, for example, in WO 2008/138131.

sALPs of the present methods can include not only the wild-type sequence of the sALPs described above, but any polypeptide having at least 50% (e.g., 55%, 60%, 65%, 70%, 75%, 80%, 85%, 86%, 87%, 88%, 89%, 90%, 91%, 92%, 93%, 94%, 95%, 96%, 97%, 98%, 99%, or more) sequence identity to these alkaline phosphatases (e.g., SEQ ID NOs: 1-19; for example the sALP fusion polypeptide of SEQ ID NO: 1 or a polypeptide variant having at least 95% sequence identity to the sequence of SEQ ID NO: 1, e.g., asfotase alfa). Examples of mutations that can be introduced into an ALP sequence are described in US Publication No. 2013/0323244, hereby incorporated by reference in its entirety. An sALP can optionally be glycosylated at any appropriate one or more amino acid residues. In addition, an sALP can have at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to any of the sALPs described herein (such as TNALP, for example the sALP fusion polypeptide of SEQ ID NO: 1 or a polypeptide variant having at least 95% sequence identity to the sequence of SEQ ID NO: 1, e.g., asfotase alfa). An sALP can have 1, 2, 3, 4, 5, 6, 7, 8, 9, 10, or more additions, deletions, or substitutions relative to any of the sALPs described herein (such as TNALP, for example the sALP fusion polypeptide of SEQ ID NO: 1 or a polypeptide variant having at least 95% sequence identity to the sequence of SEQ ID NO: 1, e.g., asfotase alfa).

sALP Fusion Polypeptides

Any of the sALPs and linkers described herein can be combined in an sALP polypeptide, e.g., an sALP polypeptide of A-sALP-B, wherein each of A and B is absent or is an amino acid sequence of at least one amino acid (such as TNALP, for example the sALP polypeptide of SEQ ID NO: 1 or a polypeptide variant having at least 95% sequence identity to the sequence of SEQ ID NO: 1, e.g., asfotase alfa). When present, A and/or B can be any linker described herein. In some sALP polypeptides, A is absent, B is absent, or A and B are both absent. The sALP polypeptides described herein can optionally include an Fc region to provide an sALP fusion polypeptide, as described herein. The sALP polypeptide can optionally include a bone-targeting moiety, as described herein. In some sALP polypeptides, a linker, e.g., a flexible linker, can be included between the bone-targeting moiety and the sALP, such as a dipeptide sequence (e.g., leucine-lysine or aspartic acid-isoleucine). Further exemplary Fc regions, linkers, and bone-targeting moieties are described below.

Any of the sALPs, linkers, and Fc regions described herein can be combined in a fusion polypeptide, e.g., a recombinant fusion polypeptide, which includes the structure Z-sALP-Y-spacer-X-Wₙ-V, Z-Wₙ-X-spacer-Y-sALP-V, Z-sALP-Y-Wₙ-X-spacer-V, and Z-Wₙ-X-sALP-Y-spacer-V (such as TNALP, for example the sALP polypeptide of SEQ ID NO: 1 or a polypeptide variant having at least 95% sequence identity to the sequence of SEQ ID NO: 1, e.g., asfotase alfa). In particular, the structure can be Z-sALP-Y-spacer-X-Wₙ-V or Z-Wₙ-X-spacer-Y-sALP-V. The sALP can be the full-length or functional fragments of ALPs, such as the soluble, extracellular domain of the ALP, as is described herein (e.g., TNALP, PALP, GCALP and IALP). Any one of X, Y, Z, and V and/or the spacer can be absent or an amino acid sequence of at least one amino acid. Wₙ can be a bone-targeting moiety, e.g., having a series of consecutive Asp or Glu residues, in which n =1 to 50, e.g., n =3-30, e.g., 5-15, e.g., 1, 2, 3, 4, 5, 6, 7, 8, 9, 10, 11, 12, 13, 14, 15, 16, 17, 18, 19, 20, 21, 22 , 23, 24, 25, 26, 27, 28, 29, 30, 31, 32, 33, 34, 35, 36, 36, 38, 39, 40, 41, 42, 43, 44, 45, 46, 47, 48, 49, or 50. The bone-targeting moiety, if present, can be positioned anywhere in the fusion polypeptide, e.g., at or near the N-terminal or C-terminal end, and/or in the linker region. For instance, the bone-targeting moiety is at the C-terminal end. sALP polypeptides and fusion polypeptides may also lack a bone-targeting moiety.

sALP fusion polypeptides described herein can be of the structure hTNALP-Fc-Dio. In particular, sALP fusion polypeptides can include an amino acid sequence of SEQ ID NO: 1 or a polypeptide variant having at least 95% sequence identity to the sequence of SEQ ID NO: 1, e.g., asfotase alfa.

Useful spacers include, but are not limited to, polypeptides comprising a Fc, and hydrophilic and flexible polypeptides able to alleviate the repulsive forces caused by the presence of the terminal highly negatively charged peptide (e.g., Wn). For example, an sALP can be a fusion polypeptide including an Fc region of an immunoglobulin at the N-terminal or C-terminal domain. An immunoglobulin molecule has a structure that is well known in the art. It includes two light chains (˜23 kD each) and two heavy chains (˜50-70 kD each) joined by inter-chain disulfide bonds. Immunoglobulins are readily cleaved proteolytically (e.g., by papain cleavage) into Fab (containing the light chain and the VH and CH1 domains of the heavy chain) and Fc (containing the CH2 and CH3 domains of the heavy chain, along with adjoining sequences). Useful Fc fragments as described herein include the Fc fragment of any immunoglobulin molecule, including IgG, IgM, IgA, IgD, or IgE, and their various subclasses (e.g., IgG-1, IgG-2, IgG-3, IgG-4, IgA-1, IgA-2), from any mammal (e.g., human). For instance, the Fc fragment is human IgG-1. The Fc fragments can include, for example, the CH2 and CH3 domains of the heavy chain and any portion of the hinge region. The Fc region can optionally be glycosylated at any appropriate one or more amino acid residues known to those skilled in the art. In particular, the Fc fragment of the fusion polypeptide has the amino acid sequence of SEQ ID NO: 20, or has at least 50% (e.g., 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more) sequence identity to SEQ ID NO: 20. Engineered, e.g., non-naturally occurring, Fc regions can be utilized in the methods described herein, e.g., as described in International Application Pub. No. WO2005/007809, which is hereby incorporated by reference. An Fc fragment as described herein can have 1, 2, 3, 4, 5, 6, 7, 8, 9, 10, 11, 12, 13, 14, 15, 16, 17, 18, 19, 20, 25, 30, 35, 40, 50, or more additions, deletions, or substitutions relative to any of the Fc fragments described herein. The sALP fusion polypeptides described herein (such as TNALP, for example the sALP polypeptide of SEQ ID NO: 1 or a polypeptide variant having at least 95% sequence identity to the sequence of SEQ ID NO: 1, e.g., asfotase alfa) can include a peptide linker region between the Fc fragment. In addition, a peptide linker region can be included between the Fc fragment and the optional bone-targeting moiety. The linker region can be of any sequence and length that allows the sALP to remain biologically active, e.g., not sterically hindered. Exemplary linker lengths are between 1 and 200 amino acid residues, e.g., 1-5, 6-10, 11-15, 16-20, 21-25, 26-30, 31-35, 36-40, 41-45, 46-50, 51-55, 56-60, 61-65, 66-70, 71-75, 76-80, 81-85, 86-90, 91-95, 96-100, 101-110, 111-120, 121-130, 131-140, 141-150, 151-160, 161-170, 171-180, 181-190, or 191-200 amino acid residues. For instance, linkers include or consist of flexible portions, e.g., regions without significant fixed secondary or tertiary structure. Exemplary flexible linkers are glycine-rich linkers, e.g., containing at least 50%, 60%, 65%, 70%, 75%, 80%, 85%, 90%, 95%, or even 100% glycine residues. Linkers can also contain, e.g., serine residues. In some cases, the amino acid sequence of linkers consists only of glycine and serine residues. A linker can optionally be glycosylated at any appropriate one or more amino acid residues. Additionally, a linker as described herein can include any other sequence or moiety, attached covalently or non-covalently. The linker can also be absent, in which the Fc fragment and the sALP are fused together directly, with no intervening residues. Certain Fc-sALP or sALP-Fc fusion polypeptides can be viewed, according to the present disclosure, either as 1) having no linker, or as 2) having a linker which corresponds to a portion of the sALP. For example, Fc fused directly to hsTNALP (1-502) can be viewed, e.g., either as having no linker, in which the hsTNALP is amino acids 1-502, or as having a 17-amino acid linker, in which the hsTNALP (18-502).

Additional amino acid residues can be introduced into the polypeptide according to the cloning strategy used to produce the fusion polypeptides. For instance, the additional amino acid residues do not provide an additional GPI anchoring signal so as to maintain the polypeptide in a soluble form. Furthermore, any such additional amino acid residues, when incorporated into the polypeptide described herein, do not provide a cleavage site for endoproteases of the host cell. The likelihood that a designed sequence would be cleaved by the endoproteases of the host cell can be predicted as described, e.g., by Ikezawa (Biol. Pharm. Bull. 25: 409-417, 2002).

The sALPs and sALP fusion polypeptides described herein (such as TNALP, for example the sALP polypeptide of SEQ ID NO: 1 or a polypeptide variant having at least 95% sequence identity to the sequence of SEQ ID NO: 1, e.g., asfotase alfa) can be associated into dimers or tetramers. For example, two sALP-Fc monomers can covalently be linked through two disulfide bonds located in the hinge regions of the Fc fragments. Additionally, the polypeptides or fusion polypeptides described herein (e.g., an sALP polypeptide or fusion polypeptide) can be glycosylated or PEGylated.

### Production of Nucleic Acids and Polypeptides

The nucleic acids encoding sALPs and sALP fusion polypeptides (such as TNALP, for example the sALP fusion polypeptide of SEQ ID NO: 1 or a polypeptide variant having at least 95% sequence identity to the sequence of SEQ ID NO: 1, e.g., asfotase alfa) can be produced by any method known in the art. Typically, a nucleic acid encoding the desired fusion polypeptide is generated using molecular cloning methods, and is generally placed within a vector, such as a plasmid or virus. The vector is used to transform the nucleic acid into a host cell appropriate for the expression of the fusion polypeptide. Representative methods are disclosed, for example, in Maniatis et al. (Cold Springs Harbor Laboratory, 1989). Many cell types can be used as appropriate host cells, although mammalian cells are preferable because they are able to confer appropriate post-translational modifications. Host cells can include, e.g., Chinese Hamster Ovary (CHO) cell, L cell, C127 cell, 3T3 cell, BHK cell, COS-7 cell or any other suitable host cell known in the art. For example, the host cell is a Chinese Hamster Ovary (CHO) cell (e.g., a CHO-DG44 cell).

The sALPs and sALP fusion polypeptides (such as TNALP, for example the sALP fusion polypeptide of SEQ ID NO: 1 or a polypeptide variant having at least 95% sequence identity to the sequence of SEQ ID NO: 1, e.g., asfotase alfa) can be produced under any conditions suitable to effect expression of the sALP polypeptide in the host cell. Such conditions include appropriate selection of a media prepared with components such as a buffer, bicarbonate and/or HEPES, ions like chloride, phosphate, calcium, sodium, potassium, magnesium, iron, carbon sources like simple sugars, amino acids, potentially lipids, nucleotides, vitamins and growth factors like insulin; regular commercially available media like alpha-MEM, DMEM, Ham's-F12, and IMDM supplemented with 2-4 mM L-glutamine and 5% Fetal bovine serum; regular commercially available animal protein free media like Hyclone™ SFM4CHO, Sigma CHO DHFR⁻, Cambrex POWER™ CHO CD supplemented with 2-4 mM L-glutamine. These media are desirably prepared without thymidine, hypoxanthine and L-glycine to maintain selective pressure, allowing stable protein-product expression.

### Pharmaceutical Compositions and Formulations

A composition that can be used in the methods described herein (e.g., including an sALP or sALP fusion polypeptide, such as TNALP, for example the sALP polypeptide of SEQ ID NO: 1 or a polypeptide variant having at least 95% sequence identity to the sequence of SEQ ID NO: 1, e.g., asfotase alfa) can be administered by a variety of methods known in the art. As will be appreciated by the skilled artisan, the route and/or mode of administration will vary depending upon the desired results. The route of administration can depend on a variety of factors, such as the environment and therapeutic goals. In particular, the polypeptides and fusion polypeptides described herein can be administration by any route known in the art, e.g., subcutaneous (e.g., by subcutaneous injection), intravenously, orally, nasally, intramuscularly, sublingually, intrathecally, or intradermally. By way of example, pharmaceutical compositions that can be used in the methods described herein can be in the form of a liquid, solution, suspension, pill, capsule, tablet, gelcap, powder, gel, ointment, cream, nebulae, mist, atomized vapor, aerosol, or phytosome.

Dosage

Any amount of a pharmaceutical composition (e.g., including an sALP or sALP fusion polypeptide, such as TNALP, for example the sALP polypeptide of SEQ ID NO: 1 or a polypeptide variant having at least 95% sequence identity to the sequence of SEQ ID NO: 1, e.g., asfotase alfa) can be administered to an HPP patient, such as a child having HPP of about 5 years of age to about 12 years of age, an adolescent having HPP of about 13 years of age to about 17 years of age, or an adult having HPP of greater than about 18 years of age. The dosages will depend on many factors including the mode of administration and the age of the patient. For example, the sALP polypeptides (such as TNALP, for example the sALP polypeptide of SEQ ID NO: 1 or a polypeptide variant having at least 95% sequence identity to the sequence of SEQ ID NO: 1, e.g., asfotase alfa) described herein can be administered to an HPP patient, such as a child having HPP of about 5 years of age to about 12 years of age, an adolescent having HPP of about 13 years of age to about 17 years of age, or an adult having HPP of greater than about 18 years of age, in individual doses ranging, e.g., from 0.01 mg/kg to 500 mg/kg of the patient (e.g., from 0.05 mg/kg to 500 mg/kg, from 0.1 mg/kg to 20 mg/kg, from 5 mg/kg to 500 mg/kg, from 0.1 mg/kg to 100 mg/kg, from 10 mg/kg to 100 mg/kg, from 0.1 mg/kg to 50 mg/kg, 0.5 mg/kg to 25 mg/kg, 1.0 mg/kg to 10 mg/kg, 1.5 mg/kg to 5 mg/kg, or 2.0 mg/kg to 3.0 mg/kg) or from 1 μg/kg to 1,000 μg/kg (e.g., from 5 μg/kg to 1,000 μg/kg, from 1 μg/kg to 750 μg/kg, from 5 μg/kg to 750 μg/kg, from 10 μg/kg to 750 μg/kg, from 1 μg/kg to 500 μg/kg, from 5 μg/kg to 500 μg/kg, from 10 μg/kg to 500 μg/kg, from 1 μg/kg to 100 μg/kg, from 5 μg/kg to 100 μg/kg, from 10 μg/kg to 100 μg/kg, from 1 μg/kg to 50 μg/kg, from 5 μg/kg to 50 μg/kg, or from 10 μg/kg to 50 μg/kg of the patient).

Exemplary doses of an sALP include, e.g., 0.01, 0.05, 0.1, 0.5, 1, 2, 2.5, 5, 10, 20, 25, 50, 100, 125, 150, 200, 250, or 500 mg/kg; or 1, 2, 2.5, 5, 10, 20, 25, 50, 100, 125, 150, 200, 250, 500, 750, 900, or 1,000 μg/kg. In particular, compositions (e.g., including sALP (such as TNALP, for example the sALP polypeptide of SEQ ID NO: 1 or a polypeptide variant having at least 95% sequence identity to the sequence of SEQ ID NO: 1, e.g., asfotase alfa)) in accordance with the present disclosure can be administered to patients in doses ranging from about 0.001 mg/kg/day to about 500 mg/kg/day, about 0.01 mg/kg/day to about 100 mg/kg/day, or about 0.01 mg/kg/day to about 20 mg/kg/day. For example, the sALP compositions (such as TNALP, for example the sALP polypeptide of SEQ ID NO: 1 or a polypeptide variant having at least 95% sequence identity to the sequence of SEQ ID NO: 1, e.g., asfotase alfa) can be administered to patients in a weekly dosage ranging, e.g., from about 0.5 mg/kg/week to about 140 mg/kg/week, e.g., about 0.8 mg/kg/week to about 50 mg/kg/week, or about 1 mg/kg/week to about 10 mg/kg/week (e.g., about 6 or about 9 mg/kg/week). In particular, the sALP (such as TNALP, for example the sALP polypeptide of SEQ ID NO: 1 or a polypeptide variant having at least 95% sequence identity to the sequence of SEQ ID NO: 1, e.g., asfotase alfa) can be administered at a dosage of 2 mg/kg three times a week (total dose 6 mg/kg/week), 1 mg/kg six times a week (total dose 6 mg/kg/week), 3 mg/kg three times a week (total dose 9 mg/kg/week), 0.5 mg/kg three times a week (total dose of 1.5 mg/kg/week), or 9.3 mg/kg three times a week (total dose 28 mg/kg/week). The dosage will be adapted by the clinician in accordance with conventional factors such as the extent of the disease and different parameters from the HPP patient, such as such as a child having HPP of about 5 years of age to about 12 years of age, an adolescent having HPP of about 13 years of age to about 17 years of age, or an adult having HPP of greater than about 18 years of age.

Dosages of compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be provided in either a single or multiple dosage regimens. Doses can be administered, e.g., hourly, bihourly, daily, bidaily, twice a week, three times a week, four times a week, five times a week, six times a week, weekly, biweekly, monthly, bimonthly, or yearly. Alternatively, doses can be administered, e.g., twice, three times, four times, five times, six times, seven times, eight times, nine times, 10 times, 11 times, or 12 times per day. In particular, the dosing regimen is once weekly. The duration of the dosing regimen can be, e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 day(s), week(s), or month(s), or even for the remaining lifespan of the HPP patient, such as a child having HPP of about 5 years of age to about 12 years of age, an adolescent having HPP of about 13 years of age to about 17 years of age, or an adult having HPP of greater than about 18 years of age. The amount, frequency, and duration of dosage will be adapted by the clinician in accordance with conventional factors such as the extent of the disease and different parameters from the HPP patient, such as a child having HPP of about 5 years of age to about 12 years of age, an adolescent having HPP of about 13 years of age to about 17 years of age, or an adult having HPP of greater than about 18 years of age.

For example, an sALP or sALP fusion polypeptide (such as TNALP, for example the sALP polypeptide of SEQ ID NO: 1 or a polypeptide variant having at least 95% sequence identity to the sequence of SEQ ID NO: 1, e.g., asfotase alfa) can be formulated as a solution for injection, which is a clear, colorless to slightly yellow, aqueous solution, pH 7.4. The sALP or sALP polypeptide (such as TNALP, for example the sALP polypeptide of SEQ ID NO: 1 or a polypeptide variant having at least 95% sequence identity to the sequence of SEQ ID NO: 1, e.g., asfotase alfa) may be formulated at a concentration of 12 mg/0.3 mL,18 mg/0.45 mL, 28 mg/0.7 mL, 40 mg/1m1, or 80 mg/0.8 mL. In particular, the composition can be formulated as a 40 mg/ml solution for injection, in which each ml of solution contains 40 mg of sALP or sALP polypeptide (e.g., each vial contains 0.3 ml solution and 12 mg of sALP (40 mg/ml), each vial contains 0.45 ml solution and 18 mg of sALP (40 mg/ml), each vial contains 0.7 ml solution and 28 mg of sALP (40 mg/ml), or each vial contains 1.0 ml solution and 40 mg of asfotase alfa (40 mg/ml)). An sALP or sALP polypeptide (such as TNALP, for example the sALP polypeptide of SEQ ID NO: 1 or a polypeptide variant having at least 95% sequence identity to the sequence of SEQ ID NO: 1, e.g., asfotase alfa) can be formulated as a solution for injection at a concentration of 100 mg/ml, in which each 1 ml of solution contains 100 mg of sALP or sALP polypeptide (e.g., each vial contains 0.8 ml solution and 80 mg of asfotase alfa (100 mg/ml)). The volume of the sALP injected in the patient may be, e.g., 0.15 ml, 0.18 ml, 0.20 ml, 0.23 ml, 0.25 ml, 0.28 ml, 0.30 ml, 0.33 ml, 0.35 ml, 0.38 ml, 0.40 ml, 0.43 ml, 0.45 ml, 0.48 ml, 0.50 ml, 0.63 ml, 0.75 ml, 0.88 ml, or 1.00 ml.

For example, the recommended dosage of an sALP or sALP fusion polypeptide ((such as TNALP, for example the sALP polypeptide of SEQ ID NO: 1 or a polypeptide variant having at least 95% sequence identity to the sequence of SEQ ID NO: 1, e.g., asfotase alfa) is 2 mg/kg of body weight administered subcutaneously three times per week, or a dosage regimen of 1 mg/kg of body weight administered subcutaneously six times per week. Additional dosage information is provided below (Table 2). In particular, a 40 kg patient administered a dosage of 6 mg/kg/week would receive an injection of 80 mg of the sALP in 0.8 ml three times a week or 40 mg of the sALP in 1.00 ml six times a week, while a 50 kg patient administered a dosage of 6 mg/kg/week would receive an injection of 50 mg of the sALP in 0.05 ml six times a week.

TABLE 2
DOSING OF ASFOTASE ALFA

| | If injecting 3x per week | | | | | If injecting 6 x per week | | | | |
|---|---|---|---|---|---|---|---|---|---|---|
| Body | Dose | | Volume | Vial type | | Dose | | Volume | Vial type | |
| Weight | to be | | to be | used for | | to be | | to be | used for | |
| (kg) | injected | | injected | injection | | injected | | injected | injection | |
| 3 | 6 | mg | 0.15 ml | 0.3 | ml | | | | | |
| 4 | 8 | mg | 0.20 ml | 0.3 | ml | | | | | |
| 5 | 10 | mg | 0.25 ml | 0.3 | ml | | | | | |
| 6 | 12 | mg | 0.30 ml | 0.3 | ml | 6 | mg | 0.15 ml | 0.3 | ml |
| 7 | 14 | mg | 0.35 ml | 0.45 | ml | 7 | mg | 0.18 ml | 0.3 | ml |
| 8 | 16 | mg | 0.40 ml | 0.45 | ml | 8 | mg | 0.20 ml | 0.3 | ml |
| 9 | 18 | mg | 0.45 ml | 0.45 | ml | 9 | mg | 0.23 ml | 0.3 | ml |
| 10 | 20 | mg | 0.50 ml | 0.7 | ml | 10 | mg | 0.25 ml | 0.3 | ml |
| 11 | 22 | mg | 0.55 ml | 0.7 | ml | 11 | mg | 0.28 ml | 0.3 | ml |
| 12 | 24 | mg | 0.60 ml | 0.7 | ml | 12 | mg | 0.30 ml | 0.3 | ml |
| 13 | 26 | mg | 0.65 ml | 0.7 | ml | 13 | mg | 0.33 ml | 0.45 | ml |
| 14 | 28 | mg | 0.70 ml | 0.7 | ml | 14 | mg | 0.35 ml | 0.45 | ml |
| 15 | 30 | mg | 0.75 ml | 1 | ml | 15 | mg | 0.38 ml | 0.45 | ml |
| 16 | 32 | mg | 0.80 ml | 1 | ml | 16 | mg | 0.40 ml | 0.45 | ml |
| 17 | 34 | mg | 0.85 ml | 1 | ml | 17 | mg | 0.43 ml | 0.45 | ml |
| 18 | 36 | mg | 0.90 ml | 1 | ml | 18 | mg | 0.45 ml | 0.45 | ml |
| 19 | 38 | mg | 0.95 ml | 1 | ml | 19 | mg | 0.48 ml | 0.7 | ml |
| 20 | 40 | mg | 1.00 ml | 1 | ml | 20 | mg | 0.50 ml | 0.7 | ml |
| 25 | 50 | mg | 0.50 ml | 0.8 | ml | 25 | mg | 0.63 ml | 0.7 | ml |
| 30 | 60 | mg | 0.40 ml | 0.8 | ml | 30 | mg | 0.75 ml | 1 | ml |
| 35 | 70 | mg | 0.70 ml | 0.8 | ml | 35 | mg | 0.88 ml | 1 | ml |
| 40 | 80 | mg | 0.80 ml | 0.8 | ml | 40 | mg | 1.00 ml | 1 | ml |
| 50 | | | | | | 50 | mg | 0.50 ml | 0.8 | ml |
| 60 | | | | | | 60 | mg | 0.60 ml | 0.8 | ml |
| 70 | | | | | | 70 | mg | 0.70 ml | 0.8 | ml |
| 80 | | | | | | 80 | mg | 0.80 ml | 0.8 | ml |
| 90 | | | | | | 90 | mg | 0.90 ml | 0.8 | ml (x2) |
| 100 | | | | | | 100 | mg | 1.00 ml | 0.8 | ml (x2) |

Formulations

The compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be formulated according to standard methods. Pharmaceutical formulation is a well-established art, and is further described in, e.g., Gennaro (2000) Remington: The Science and Practice of Pharmacy, 20th Edition, Lippincott, Williams & Wilkins (ISBN: 0683306472); Ansel et al. (1999) Pharmaceutical Dosage Forms and Drug Delivery Systems, 7th Edition, Lippincott Williams & Wilkins Publishers (ISBN: 0683305727); and Kibbe (2000) Handbook of Pharmaceutical Excipients American Pharmaceutical Association, 3^{rd} Edition (ISBN: 091733096X). For instance, an sALP composition (such as TNALP, for example the sALP polypeptide of SEQ ID NO: 1 or a polypeptide variant having at least 95% sequence identity to the sequence of SEQ ID NO: 1, e.g., asfotase alfa) can be formulated, for example, as a buffered solution at a suitable concentration and suitable for storage at 2-8° C. (e.g., 4° C.). A composition can also be formulated for storage at a temperature below 0° C. (e.g., −20° C. or −80° C.). A composition can further be formulated for storage for up to 2 years (e.g., one month, two months, three months, four months, five months, six months, seven months, eight months, nine months, 10 months, 11 months, 1 year, 1½ years, or 2 years) at 2-8° C. (e.g., 4° C.). Thus, the compositions described herein can be stable in storage for at least 1 year at 2-8° C. (e.g., 4° C.).

The compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can be in a variety of forms. These forms include, e.g., liquid, semi-solid and solid dosage forms, such as liquid solutions (e.g., injectable and infusible solutions), dispersions or suspensions, tablets, pills, powders, liposomes and suppositories. The preferred form depends, in part, on the intended mode of administration and therapeutic application.

For example, compositions intended for systemic or local delivery can be in the form of injectable or infusible solutions. Accordingly, the compositions (such as TNALP, for example the sALP polypeptide of SEQ ID NO: 1 or a polypeptide variant having at least 95% sequence identity to the sequence of SEQ ID NO: 1, e.g., asfotase alfa) can be formulated for administration by a parenteral mode (e.g., subcutaneous, intravenous, intraperitoneal, or intramuscular injection).

The compositions including sALPs and sALP fusion polypeptides (such as TNALP, for example the sALP fusion polypeptide of SEQ ID NO: 1 or a polypeptide variant having at least 95% sequence identity to the sequence of SEQ ID NO: 1, e.g., asfotase alfa) can be formulated as a solution, microemulsion, dispersion, liposome, or other ordered structure suitable for stable storage at high concentration. Sterile injectable solutions can be prepared by incorporating a composition described herein in the required amount in an appropriate solvent with one or a combination of ingredients enumerated above, as required, followed by filter sterilization. Generally, dispersions are prepared by incorporating a composition described herein into a sterile vehicle that contains a basic dispersion medium and the required other ingredients from those enumerated above. In the case of sterile powders for the preparation of sterile injectable solutions, methods for preparation include vacuum drying and freeze-drying that yield a powder of a composition described herein plus any additional desired ingredient (see below) from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition a reagent that delays absorption, for example, monostearate salts, and gelatin.

The compositions described herein can also be formulated in immunoliposome compositions. Such formulations can be prepared by methods known in the art such as, e.g., the methods described in Epstein et al. (1985) Proc Natl Acad Sci USA 82:3688; Hwang et al. (1980) Proc Natl Acad Sci USA 77:4030; and U.S. Pat. Nos. 4,485,045 and 4,544,545. Liposomes with enhanced circulation time are disclosed in, e.g., U.S. Pat. No. 5,013,556.

Compositions including sALPs and sALP fusion polypeptides (such as TNALP, for example the sALP polypeptide of SEQ ID NO: 1 or a polypeptide variant having at least 95% sequence identity to the sequence of SEQ ID NO: 1, e.g., asfotase alfa) can also be formulated with a carrier that will protect the composition (e.g., an sALP polypeptide or sALP fusion polypeptide) against rapid release, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are known in the art. See, e.g., J. R. Robinson (1978) Sustained and Controlled Release Drug Delivery Systems, Marcel Dekker, Inc., New York.

When compositions are to be used in combination with a second active agent, the compositions can be co-formulated with the second agent, or the compositions can be formulated separately from the second agent formulation. For example, the respective pharmaceutical compositions can be mixed, e.g., just prior to administration, and administered together or can be administered separately, e.g., at the same or different times.

Carriers/Vehicles

Preparations containing an sALP or sALP fusion polypeptide (such as TNALP, for example the sALP polypeptide of SEQ ID NO: 1 or a polypeptide variant having at least 95% sequence identity to the sequence of SEQ ID NO: 1, e.g., asfotase alfa) can be provided to HPP patients, such as a child having HPP of about 5 years of age to about 12 years of age, an adolescent having HPP of about 13 years of age to about 17 years of age, or an adult having HPP of greater than about 18 years of age, in combination with pharmaceutically acceptable sterile aqueous or non-aqueous solvents, suspensions or emulsions. Examples of non-aqueous solvents are propylene glycol, polyethylene glycol, vegetable oil, fish oil, and injectable organic esters. Aqueous carriers include water, water-alcohol solutions, emulsions or suspensions, including saline and buffered medical parenteral vehicles including sodium chloride solution, Ringer's dextrose solution, dextrose plus sodium chloride solution, Ringer's solution containing lactose, or fixed oils. For example, the pharmaceutically acceptable carrier can include sodium chloride and/or sodium phosphate, in which the composition includes, e.g., about 150 mM sodium chloride and/or about 25 mM sodium phosphate, pH 7.4.

Intravenous vehicles can include fluid and nutrient replenishers, electrolyte replenishers, such as those based upon Ringer's dextrose, and the like. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, can be present in such vehicles. A thorough discussion of pharmaceutically acceptable carriers is available in Remington's Pharmaceutical Sciences (Mack Pub. Co., N.J. 1991).

The following examples are intended to illustrate, rather than limit, the disclosure. These studies feature the identification of the health state of patients having HPP across age groups (children having HPP of about 5 years of age to about 12 years of age, adolescents having HPP of about 13 years of age to about 17 years of age, and adults having HPP of greater than about 18 years of age or older) and assignment of a treatment regimen including administration of asfotase alfa (SEQ ID NO: 1) to the HPP patients.

### EXAMPLES

### Example 1

### Effect of Asfotase Alfa Treatment on Health States and Ambulatory Function in Patients with Hypophosphatasia (HPP)

The skeletal manifestations of HPP are often associated with ambulatory difficulties that can impact patients' health-related quality of life (HRQoL). Ambulatory function in patients with HPP can be determined by the widely used 6-minute walk test (6MWT), which has been documented to correlate with scores from the EuroQol-5D (EQ-5D) questionnaire (Lloyd A et al. Value Health 2015; 18(7):A651). In economic evaluations using the quality-adjusted life year (QALY), HRQoL is expressed as a health state utility value (HSUV). To generate HSUVs, the health states (I, II, III, or IV) to which the values are assigned need to first be defined. A previous analysis defined health states for HPP based on predicted 6MWT performance, as informed by minimally clinically important difference (MCID) values estimated from a Duchenne muscular dystrophy population. The MCID for the 6MWT in HPP was estimated to be 31 m Tomazos I et al. (Abstract presented at the Annual Meeting of the European Society for Paediatric Endocrinology, Sep. 10-12, 2016, Paris, France). This study aimed to build on the previous analysis by defining HPP-specific health states associated with three age cohorts (e.g., 5-12,13-17 and 18 years) based on 6MWT data from patients with HPP. We also aimed to determine the effect of asfotase alfa on the transition of patients between these health states and define patient profiles for each of the health states.

### Methods

Ambulation was assessed by the 6MWT in two clinical studies of astofase alfa (NCT00952484/NCT01203826, NCT01163149) in 29 patients aged 5 years with HPP. Patient 6MWT results were described as a percentage of normal function (calculated from age-, sex- and height-adjusted data from healthy individuals), with 80% defining the lower limit of normal. Mean baseline 6MWT data from each age group were used to estimate MCID values using established distribution-based methodology (one third baseline standard deviation) (McDonald C M et al. Muscle Nerve 2013; 48:357-680). The MCID values were then multiplied by a severity level step size (calculated as 2× MCID/mean 6MWT) to determine the percentage predicted 6MWT for four health states: severity levels 1 (lowest impact on ambulation), 2,3 and 4 (highest impact on ambulation) (Table 3).

TABLE 3
Percentage prediction for 6 MWT for each
health state within each age group.

| HPP | 6 MWT, % of normal functioning | | |
|---|---|---|---|
| health | Age 5-12 years | Age 13-17 years | Age ≥18 years |
| state | (n = 13) | (n = 6) | (n = 10) |
| I (lowest | >82.4 to ≤100 | >82.6 to ≤100 | >84.0 to ≤100 |
| impact on | | | |
| ambulation) | | | |
| II | >64.8 to ≤82.4 | >65.2 to ≤82.6 | >68.0 to ≤84.0 |
| III | >47.2 to 64.8 | >47.8 to ≤65.2 | >52.0 to ≤68.0 |
| IV (highest | ≤47.2 | ≤47.8 | ≤52.0 |
| impact on | | | |
| ambulation) | | | |

6MWT data at treatment initiation and at 96 weeks of treatment were assigned to a corresponding health state. Patient profiles for the four health states for each of the three age cohorts were created based on a review of the literature and an advisory board of four clinical experts with experience in treating children or adults with HPP, and were revised following open-ended interviews the same clinical experts. Additional clinical experts were then asked, via telephone interview, to describe the clinical features of HPP and complete the EQ-5D-3-Level (EQ-5D-3L) questionnaire and the Child Health Utility 9-Dimension (CHU-9D) questionnaire to reflect the impact of HPP on patients' HRQoL at each health state within each age group; this informed a second and final revision of the patient profiles.

### Results

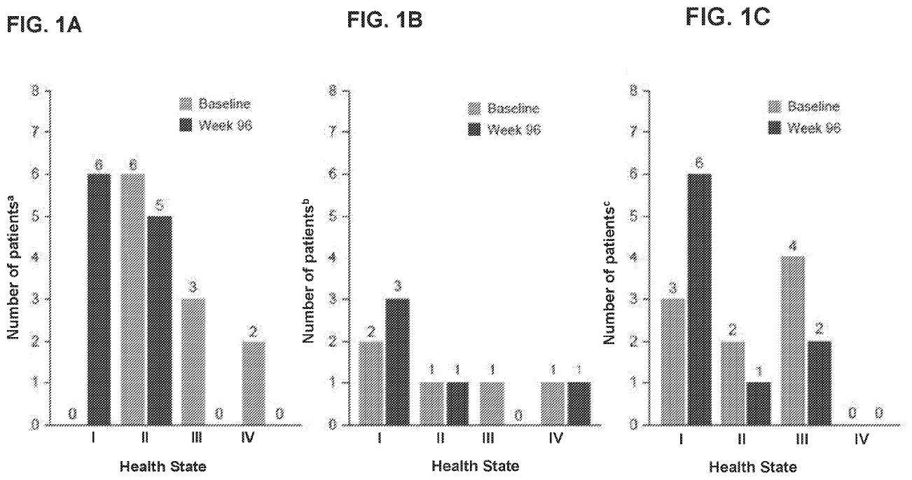

The distribution of patients between health states at baseline and after 96 weeks of treatment with asfotase alfa is shown in Table 4 and FIGS. 1A-1C.

TABLE 4
Patient distribution between health states at baseline
and at 96 weeks of asfotase alfa treatment.

| | Patients in each health state, n (%) | | | | | | | | | | | |
|---|---|---|---|---|---|---|---|---|---|---|---|---|
| | Age 5-12 years | | | | Age 13-17 years | | | | Age ≥18 years | | | |
| HPP | (n = 11) | | | | (n = 5) | | | | (n = 9) | | | |
| health state | Baseline | | Week 96 | | Baseline | | Week 96 | | Baseline | | Week 96 | |
| I (lowest | 0 | (0) | 6 | (54.5) | 2 | (40.0) | 3 | (60.0) | 3 | (33.3) | 6 | (66.7) |
| impact on | | | | | | | | | | | | |
| ambulation) | | | | | | | | | | | | |
| II | 6 | (54.5) | 5 | (45.5) | 1 | (20.0) | 1 | (20.0) | 2 | (22.2) | 1 | (11.1) |
| III | 3 | (27.3) | 0 | (0) | 1 | (20.0) | 0 | (0) | 4 | (44.4) | 2 | (22.2) |
| IV (highest | 2 | (18.2) | 0 | (0) | 1 | (20.0) | 1 | (20.0) | 0 | (0) | 0 | (0) |
| impact on | | | | | | | | | | | | |
| ambulation) | | | | | | | | | | | | |

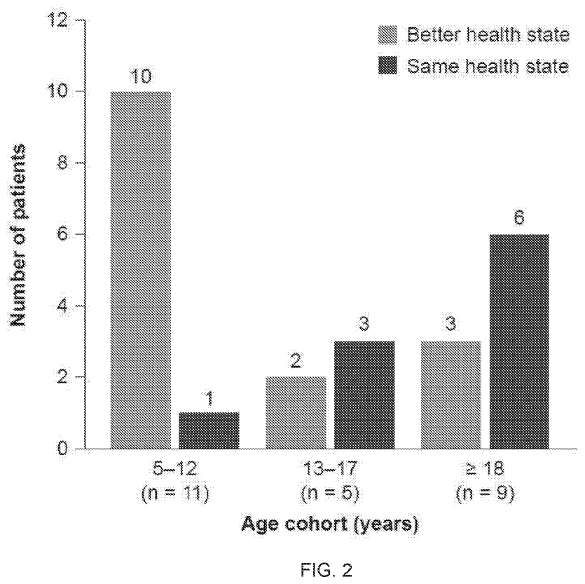

In general, following 96 weeks of asfotase alfa therapy all patients with HPP either transitioned to a better health state or remained in the health state to which they were assigned before treatment was initiated; no patients transitioned to a worse health state (Table 5, FIG. 2).

TABLE 5
Transition of patients between health states from
baseline to 96 weeks of asfotase alfa treatment.

| | Transition to another health state at 96 | | | | | |
|---|---|---|---|---|---|---|
| | weeks of treatment, n (%) | | | | | |
| | Age 5-12 years | | Age 13-17 years | | Age ≥18 years | |
| Transition | (n = 11) | | (n = 5) | | (n = 9) | |
| Milder health state | 10 | (91) | 2 | (40) | 3 | (33) |
| Same health state | 1 | (9) | 3 | (60) | 6 | (67) |
| Worse health state | 0 | (0) | 0 | (0) | 0 | (0) |

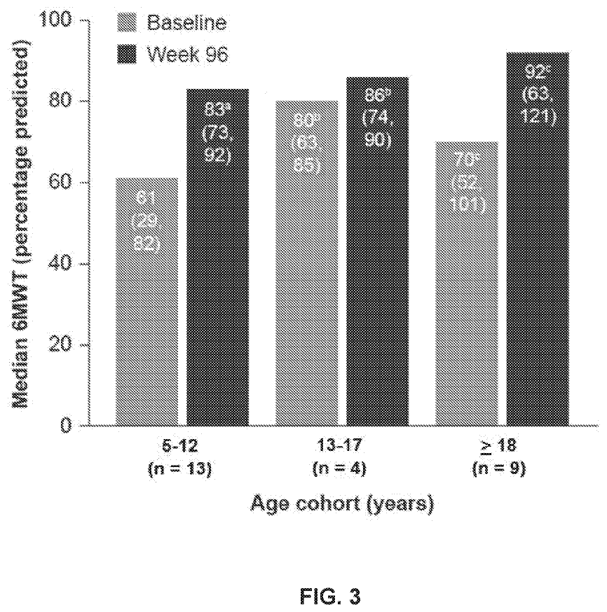

Across all three age groups, an improvement in median 6MWT from baseline to 96 weeks of treatment with asfotase alfa was observed (Table 6, FIG. 3).

TABLE 6
Median patient 6 MWT results at baseline and
at 96 weeks of asfotase alfa treatment.

| | Group 6 MWT results, % of normal function | | | | | |
|---|---|---|---|---|---|---|
| | Age 5-12 years | | Ages 13-17 years | | Age ≥18 years | |
| | Baseline | Week 96 | Baseline | Week 96 | Baseline | Week 96 |
| | (n = 13) | (n = 11) | (n = 4) | (n = 4) | (n = 10) | (n = 9) |
| Median | 61 | 83 | 80 | 86 | 69 | 92 |
| (IQR) | (29, 82) | (73, 92) | (63, 85) | (74, 90) | (42,101) | (63,121) |

IQR = interquartile range.

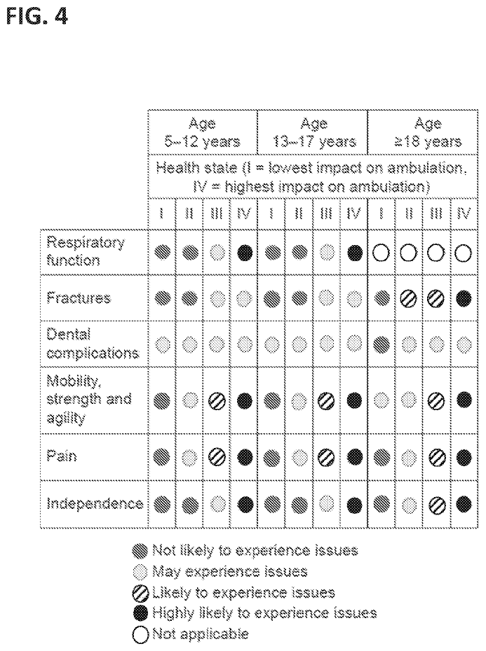

During the telephone interviews, HPP clinical experts confirmed that the revised 12 patient profiles (one for each of the four health states for each of the three age cohorts) accurately reflected the range of HPP disease presentations observed in clinical practice. Key characteristics of each of the 12 patient profiles are shown in FIG. 4. Many descriptions, such as levels of mobility and pain, are consistent across the age groups for the same health state. However, some features are characteristic of HPP disease presentation in a certain age group; for example the incidence of prolonged respiratory infections requiring antibiotics and possibly hospitalization was only documented in the 5-12 and 13-17 year age groups at severity level IV.

### Conclusions

Treatment with asfotase alfa was associated with an improvement or no change in the health states derived from the 6MWT data for patients with HPP across all three age cohorts. No patient experienced a decrease in health state while on treatment. On average, children, adolescents, and adults with HPP achieved the normal range for the 6MWT % predicted for age (e.g., greater than 80% of the predicted 6MWT value for a healthy subject of the same age, gender, and/or height). These results show that HPP patients can be assigned to one of four health states based on metrics, such as the 6MWT and EQ-5D, and improvement or maintenance of health state can be seen as a result of treatment. While the patient profiles have been designed to represent the range of severities of HPP within different age groups, they are by definition a simplification and may not accurately reflect the clinical variability that is seen in these patients. The results from this study will be valuable in QALY assessments by providing health states to generate HSUVs, thereby overcoming issues surrounding QALY estimation in rare diseases owing to the reliance of single arm trials that make aggregation of HRQoL data extremely challenging.

### Example 2

### Frequency and Time of Clinical Symptom Onset Impacting Health-Related Quality-of-Life Dimensions in Patients with HPP

HPP is a heterogeneous disease, with the age at sign/symptoms onset of HPP ranging from in utero to adulthood and the potential involvement of multiple systems. Perinatal and infantile onset HPP are associated with significant mortality; however, many patients with HPP will experience progression of their disease, and the disabling clinical impacts of this condition. These include delayed motor milestones, muscle weakness, ambulatory/functional difficulties, pain, dental abnormalities, and frequent low trauma fractures. A thorough understanding of the clinical course of HPP is limited by the rarity of the condition. No robust study of the natural history has yet been published and most evidence is available in the form of case reports. To the best of our knowledge, there are no large clinical case series or long-term observational studies documenting clinical progression and outcomes in patients with HPP. Even less is known about the health-related quality-of-life (HRQoL) in patients with HPP, given that few data describing HRQoL have been presented in published case reports. A synthesis of available clinical data on symptoms and events with likely HRQoL impacts, from cases with longitudinal follow-up over time would be valuable for characterizing the HRQoL effects experienced by patients with HPP. The objective of this study was to estimate the occurrence of, and time to, key clinical symptoms and events which impact HRQoL from a broad range of HPP patients from the published literature using a novel approach to synthetizing case report data.

### Methods

The conduct of the study and interpretation of the findings of the analyses was guided by physicians with clinical experience in managing patients with HPP. A systematic review was conducted in PubMed/Medline and EMBASE to identify HPP cases with longitudinal (≥1 year) follow up, from literature database inception dates to February 2017. Eligible study designs were case series and case reports where data on individual patients were reported in sufficient detail to understand timing and frequency of clinical symptoms and events (e.g., surgeries, hospitalizations, etc.). Double screening, review, and data extraction were performed for study design, study characteristics, patient characteristics, and clinical data from eligible studies. Demographic characteristics of the longitudinal cases were summarized. Outcomes were calculated overall, and according to the age at onset of HPP. The age categories for this analysis were based on commonly used American Pediatric Associated age groupings:
In utero;
Infancy/early childhood (<2 years); and considered at age 0 to <6 months vs 6 <24 months for selected outcomes;
Childhood (ages 2 to <10);
Early adolescence/adolescence (ages 10 to <18); and
Adulthood (age 18)

In order to visualize clinical symptoms and events over time, broad symptom categories were defined based on clinical experience: skeletal, dental, gross motor, respiratory, and kidney. Individual sign or symptom codes were classified accordingly into the broad symptom categories, along with the age at occurrence. Bar charts were used to visualize patterns at the broad symptom level over time

For HRQoL analysis, clinical symptoms and events with potentially important impacts on activities of daily living, functional and emotional status, and HRQoL, were identified by consultation with clinical experts, and these data extracted along with patient age at time of occurrence. Additionally, these symptoms and events were thought to be sufficiently impactful, in that they would have been likely reported if they had occurred. Clinical symptoms with the potential to impact HRQoL were: premature loss of teeth, other dental abnormalities, fracture, pain, gross motor/ambulation difficulties, cranial abnormalities, respiratory symptoms, nephrocalcinosis, seizures, psychological, and renal failure. Events with the potential to impact HRQoL were: hospitalizations and surgeries

The median (range) times to first onset of the most frequent symptoms with HRQoL impacts were determined using Kaplan-Meier curves. Due to the underlying distribution of data, median times to respiratory and cranial abnormalities (commonly experienced by infants and young children with HPP) were calculated only from those experiencing the event. By restricting this analysis to cases with year follow up, early deaths often resulting from respiratory symptoms were excluded; this reduced the number of cases with respiratory symptoms in the current analysis.

### Results

Of 3040 screened abstracts, data were extracted from 283 case reports and case series; 511 unique cases of HPP were identified, 265 of whom had longitudinal year) follow up and were included in this analysis. 11.3% of included cases had disease onset in utero, 38.5% during infancy/early childhood (<2 years), 29.4% during childhood (2 to <10 years, 3.4% during early adolescence/adolescence (10 to <18), and 17.4% during adulthood (18 years). The demographic characteristics of the sample are presented in Table 7.

TABLE 7
Demographic and clinical characteristics of
265 cases of HPP with longitudinal follow-up.

| Characteristic | n | % |
|---|---|---|
| Total cases | 265 | 100 |
| Male sex | 118 | 44.5 |
| Median (IQR) age at anchor visit (years) | 4.0 | 0.4-36.0 |
| Known family history of HPP | 74 | 27.9 |
| Age at disease onset (years) | | |
| In utero | 30 | 11.3 |
| Infancy/early childhood (0 to <2 years) | 102 | 38.5 |
| Childhood (2 to <10 years) | 78 | 29.4 |
| Adolescence (10 to <18 years) | 9 | 3.4 |
| Adulthood (≥18 years) | 46 | 17.4 |
| Median (IQR) duration of follow-up | | |
| Overall | 7.0 | 3.0-18.0 |
| In utero | 4.0 | 2.1-10.3 |
| Infancy/early childhood (0 to <2 years) | 5.4 | 2.4-14.8 |
| Childhood (2 to <10 years) | 7.7 | 3.6-28.5 |
| Adolescence (10 to <18 years) | 6.0 | 4.3-27.0 |
| Adulthood (≥18 years) | 15.0 | 6.3-27.0 |

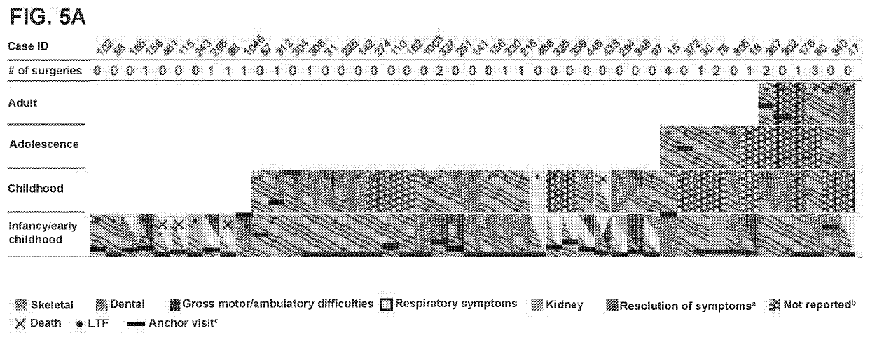

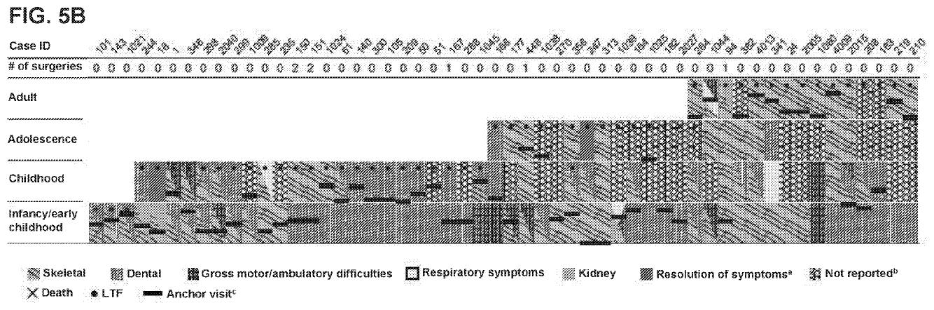

Of the 265 cases 44.5% were male and 27.9% had a known family history of HPP. The median (interquartile range; IQR) age at first reported presentation was 4.0 years and the median (IQR) available follow-up was 7.0 years (3.0-18.0). For the largest subgroup (disease onset in infancy/early childhood), a bar chart of clinical symptoms and event patterns is presented in FIGS. 5A-5B. Respiratory symptoms were more frequently observed among cases with a first HPP symptom between 0 to 6 months (27.6%, FIG. 5A) compared to those with a first HPP symptom from 6 to 24 months (3.7% FIG. 5B). Dental-only symptoms were more frequently reported among cases with a first symptom in infancy/early childhood when that first symptom occurred after 6 months (FIG. 5B). As patients progressed to adolescence and adulthood, skeletal-only symptoms were the most frequently observed for both subgroups.

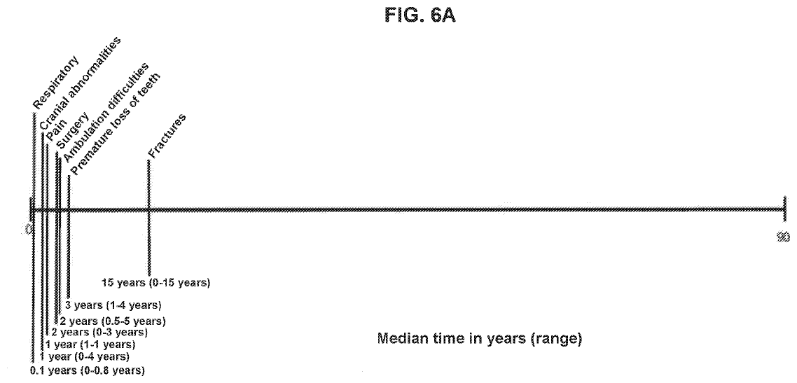

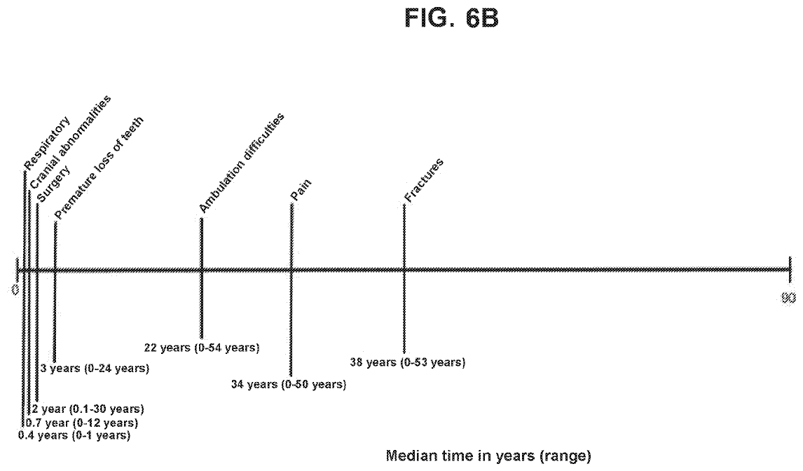

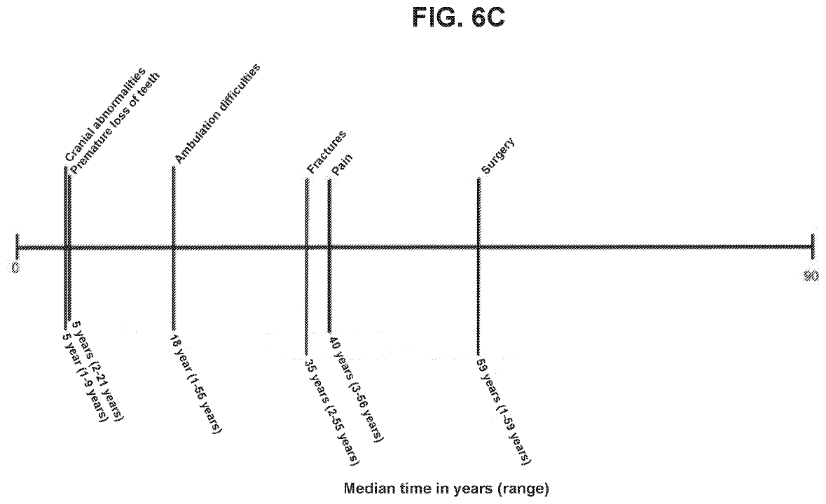

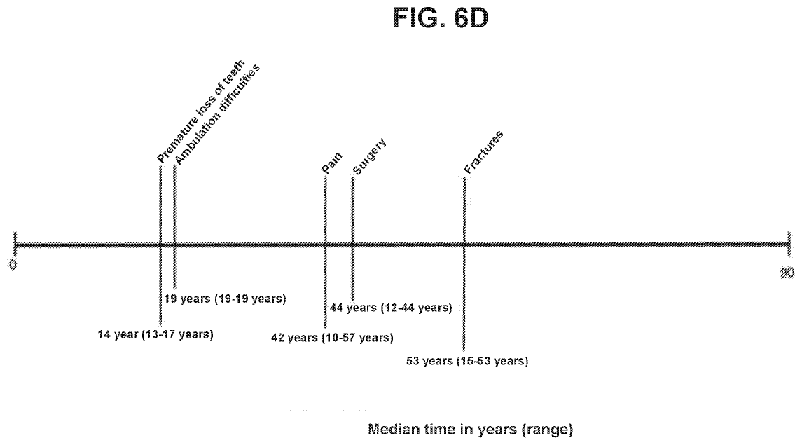

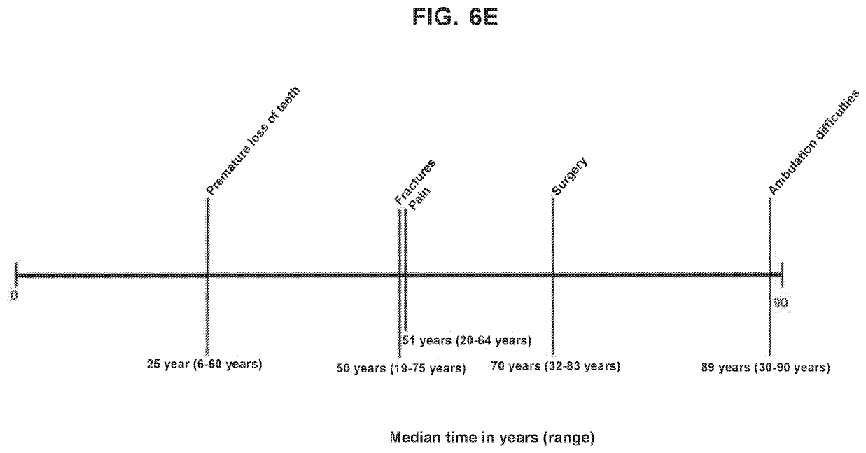

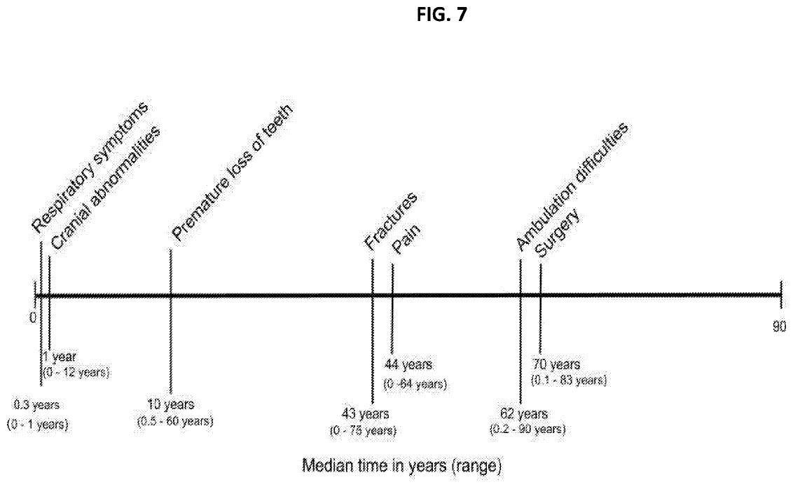

Most patients (94%) experienced at least one clinical symptom or event that could impact HRQoL over their follow up; the most frequent were premature tooth loss (53.6%), fractures (34.2%; almost half had ≥3), ambulation difficulties (29.5%), pain (32.0%), cranial abnormalities (23.7%); and surgeries (22.3%; Table 8). For each age at onset category, the median (range) years to clinical symptoms and events that can impact HRQoL are illustrated in FIGS. 6A-6E.

TABLE 8
Frequency (%) of cases with HRQoL impacting symptoms,
overall and by age at disease onset.

| Symptom with | Overall | In utero | Infancy | Childhood | Adolescence | Adulthood |
|---|---|---|---|---|---|---|
| HRQoL impact | (n = 265) | (n = 30) | (n = 102) | (n = 78) | (n = 9) | (n = 46) |
| Premature loss | [symbol] | 10.0 | [symbol] | [symbol] | [symbol] | 30.4 |
| of teeth | | | | | | |
| Fracture | [symbol] | [symbol] | 18.6 | [symbol] | [symbol] | [symbol] |
| Pain | [symbol] | 3.3 | 24.5 | [symbol] | [symbol] | [symbol] |
| Gross motor/ | 29.5 | [symbol] | [symbol] | 23.1 | 0.0 | 32.6 |
| ambulation | | | | | | |
| difficulties | | | | | | |
| Cranial | 23.7 | [symbol] | [symbol] | 14.1 | 0.0 | 0.0 |
| abnormalities | | | | | | |
| Surgeries | 22.3 | 13.3 | 29.4 | 10.3 | [symbol] | [symbol] |
| Dental other^{a} | 13.3 | 0.0 | 22.5 | 14.1 | 0.0 | 6.5 |
| Hospitalizations | 11.9 | 3.3 | 17.6 | 7.7 | 22.2 | 13.0 |
| Respiratory | 6.8 | 16.7 | 13.7 | 0.0 | 0.0 | 0.0 |
| symptoms | | | | | | |
| Nephrocalcinosis | 4.0 | 6.7 | 7.8 | 1.3 | 0.0 | 0.0 |
| Seizures | 2.5 | 10.0 | 2.9 | 0.0 | 11.1 | 2.2 |
| Psychological | 1.1 | 0.0 | 0.0 | 1.3 | 0.0 | 4.3 |
| Renal failure | 1.4 | 0.0 | 1.0 | 3.8 | 0.0 | 0.0 |

Bolded italicized numbers represent the three most frequent symptoms in each column
^{a}Dental other includes delayed dentition, abnormal dentition, advanced periodontitis, and alveolar bone loss

With the exception of cranial abnormalities and respiratory symptoms, the risk of HRQoL impacts increased with increasing age (FIGS. 6A-6E). For the sample overall, the median (range) years to clinical symptoms and events that can impact HRQOL were: respiratory difficulties, 0.3 (0-1); cranial abnormalities, 1 (0-13); premature tooth loss, 10 (0.5-60); fracture, 43 (0-75); pain, 44 (0-64); ambulation difficulties, 62 (0.2-90); and surgery, 70 (0.1-83; FIG. 7).

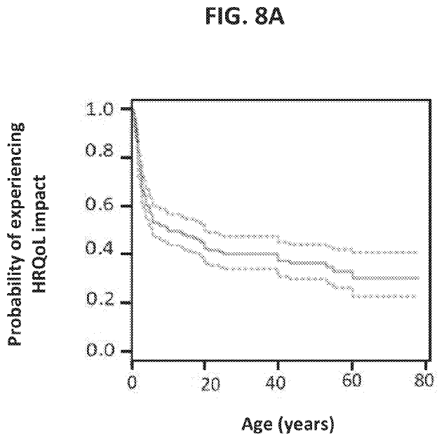

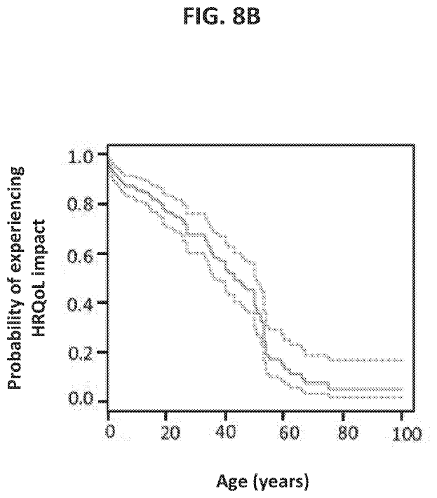

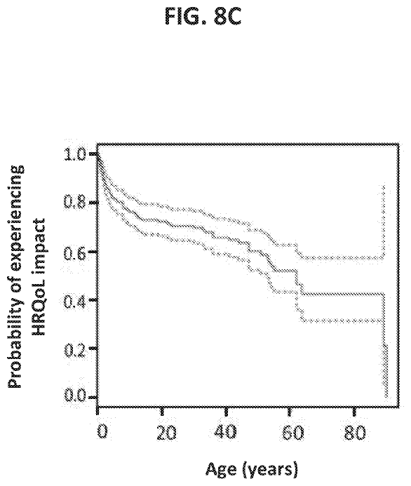

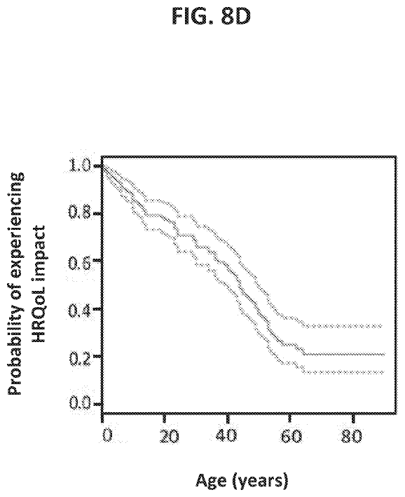

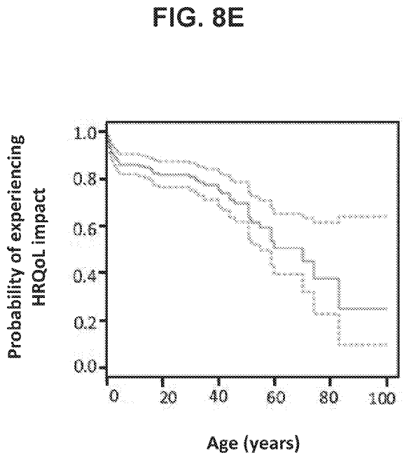

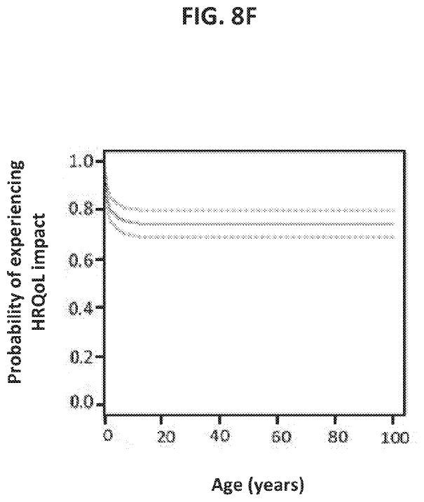

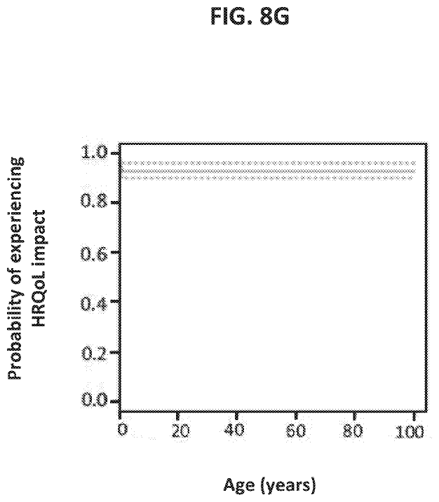

Kaplan-Meier curves illustrating the time to these events for the overall sample are presented in FIGS. 8A-8G. Cranial abnormalities and respiratory symptoms occurred almost exclusively among in utero and infancy/early childhood onset patients with HPP. Premature loss of teeth was common in childhood, but also occurred throughout adolescence and adulthood (e.g. the premature loss of permanent teeth). The median times to fracture, gross motor, pain, and surgery were consistent with incidence in adulthood (62, 44, and 70 years respectively).

### Study Limitations

The use of case report data has limitations; including that clinical data are not routinely collected at regular follow-up visits. A required assumption was that the absence of reporting of clinical symptoms and events meant that those symptoms and events had not occurred. For this reason, the primary focus was major clinical symptoms and events (e.g. cranial abnormalities, fracture, and surgery), which would be more likely to be reported and likely to have manifest as some burden to the patient. While these methods cannot replace well designed and conducted natural history studies, they help to address a gap in our understanding of disease progression and HRQoL impacts in patients with HPP. For the purposes of observing frequency and time to clinical symptoms and events, individual symptoms and events were grouped into broader categories (e.g., mild and severe gross motor problems considered together). While this compromises the ability to draw case-level conclusions from the data, this was required to facilitate the characterization of symptom and event patterns over time

### Conclusions

Understanding the natural history and burden of HPP from both a clinical and HRQoL perspective is challenging because of the rarity of the disease and the limited availability of rigorously-collected data. Directly-reported HRQoL data were not commonly reported in the case reports. Based on clinical guidance the importance of the symptoms and events that would likely have HRQoL impacts were inferred. Almost 95% of cases included at least one HPP symptom or event identified as likely having an impact on HRQoL; the most frequently observed HRQoL-impacting symptoms across ages were premature tooth loss (in one-half of cases) and fractures and ambulation difficulties (in one-third of cases).

The consistency with which HRQoL symptoms impact HPP patients of different ages suggest that, regardless of clinical classifications of HPP used in the current literature (HPP-onset), all patients with HPP are likely to develop symptoms and events that impact HRQoL over time. In particular, as a patient ages, they develop pain, fractures, experience ambulation difficulties, and undergo a number of surgeries. The evolution of HPP over time is associated with the development of symptoms and events with significant impacts on HRQoL in patients of all ages. Cranial abnormalities and respiratory symptoms were experienced by cases in infancy and early childhood; premature loss of teeth was common in childhood but also occurred throughout adulthood.

Patient profiles for the four health states for each of the three age cohorts can be created based on a review of the literature and an advisory board of clinical experts with experience in treating children or adults with HPP. These health states may be revised following open-ended interviews from the same clinical experts. Additional clinical experts may then be asked (e.g., via telephone interview) to describe the clinical features of HPP and complete a quality of life assessment (e.g., EQ-5D-3-Level (EQ-5D-3L), or Child Health Utility 9-Dimension (CHU-9D)) questionnaire to reflect the impact of HPP on patients' HRQoL at each health state within each age group. This may inform a second and final revision of the patient profiles. The data from other quality of life assessments can be combined with the data synthesized in this study to most accurately estimate and identify the occurrence of, and time to, key clinical symptoms and events which impact HRQoL for a broad range of HPP patients, based on, in part, when the key symptoms and events occur in life. For example, the EQ-5D can be used to assess the presence of symptoms and the severity of the symptoms associated with a particular health state (e.g., I-IV). When combined with a second assessment, such as the 6MWT, the EQ-5D can also be used to determine whether an HPP patient has improved from one health state to another, e.g., following treatment with asfotase alfa.

Median time to fractures, pain, ambulation difficulties, and surgeries were all in adulthood suggesting that these are indeed experienced by HPP patients of all ages, and can occur over a lifetime. In general, these clinical symptoms and events were frequent and can substantially impact HRQoL. The risk of occurrence of these clinical symptoms and events increases with age.

### Other Embodiments

All publications, patents, and patent applications mentioned in the above specification are hereby incorporated by reference to the same extent as if each individual publication, patent or patent application was specifically and individually indicated to be incorporated by reference in its entirety. Various modifications and variations of the described methods, pharmaceutical compositions, and kits described herein will be apparent to those skilled in the art without departing from the scope and spirit of the claimed invention. Although the disclosure has been described in connection with specific embodiments, it will be understood that it is capable of further modifications and that the invention as claimed should not be unduly limited to such specific embodiments.

1. A method of assigning one of four health states to a patient with hypophosphatasia (HPP) comprising:
(a) characterizing the physiological condition of the patient using one or more metrics; and
(b) using results of the one or more metrics to assign the patient into one of the four health states.

2. A method of assigning a treatment regimen based on a health state of a patient with hypophosphatasia (HPP) comprising:
(a) characterizing the physiological condition of the patient using one or more metrics;
(b) using results of the one or more metrics to identify the health state of the patient, wherein the health state is I, II, III, or IV;
(c) assigning the treatment regimen based on the health state of the patient; and
(d) assessing a change in the health state of the patient by repeating steps (a) and (b) one or more times after completion of a treatment regimen,
wherein the treatment regimen comprises administering at least 0.5 mg/kg/week of a soluble alkaline phosphatase (sALP) to the patient for a treatment period of at least three months, wherein the sALP comprises an amino acid sequence having at least 95% sequence identity to the amino acid sequence of SEQ ID NO: 1.

3-4. (canceled)

5. The method of claim 2, wherein the treatment regimen improves the health state of the patient, particularly from IV to III, II, or I; or from III to II or I; or from II to I, or wherein the treatment regimen maintains the health state of the patient.

6. (canceled)

7. The method of claim 2, wherein the health state of the patient is characterized with at least one physical assessment selected from one or more of the following metrics: Six Minute Walk Test (6MWT), Bruininks-Oseretsky Test of Motor Proficiency 2nd Edition (BOT-2), Bayley Scales of Infant and Toddler Development, 3rd Edition (BSID-III), and gait analysis, particularly 6MWT; and at least one quality of life assessment selected from one or more of the following metrics: EuroQol Five Dimension Questionnaire (EQ-5D), Childhood Health Assessment Questionnaire (CHAQ), Pediatric Outcomes Data Collection Instrument (PODCI), Child Health Utility Index-9D (CHU-9D), Pediatric Quality of Life Inventory (PedsQL), Short Form Health Survey 36 (SF-36), and Short Form Health Survey 12 (SF-12), particularly EQ-5D.

8. The method of claim 2, wherein:
(a) the patient is about 5 years of age to about 12 years of age and the health state of the patient is characterized by performing at least one physical assessment selected from the following: 6MWT, BOT-2, gait analysis, and BSID-III, and at least one quality of life assessment selected from the following: EQ-5D, CHAQ, PODCI, CHU-9D, and PedsQL; or
(b) prior to administration of the sALP:
(i) the patient is identified as having health state IV when the patient has a 6MWT value of less than about 47.2% of a predicted 6MWT value for a healthy subject;
(ii) the patient is identified as having health state III when the patient has a 6MWT value of about 47.2% to about 64.8% of a predicted 6MWT value for a healthy subject;
(iii) the patient is identified as having health state II when the patient has a 6MWT value of about 64.8% to about 82.4% of a predicted 6MWT value for a healthy subject; or
(iv) the patient is identified as having health state I when the patient has a 6MWT value of greater than 82.4% of a predicted 6MWT value for a healthy subject; and
wherein, optionally, after administration of the sALP, the patient has an improved health state.

9-10. (canceled)

11. The method of claim 7, wherein the method further comprises assessing one or more of the following symptoms: elevated blood or urine levels of PPi, PEA, or PLP; rickets, rachitic ribs, one or more skeletal deformities, hypotonia, muscle weakness, rheumatoid complications, arthritis, pseudogout, waddling gait, ambulatory difficulties, bone pain, pain, premature loss of teeth, hypomineralization, delayed motor development, seizures, hypercalciuria, short stature, bone fracture, pseudofracture, and growth delay.

12. The method of claim 2, wherein:
(a) the patient is about 13 years of age to about 17 years of age and the health state of the patient is characterized by performing one or more of the following: 6MWT, EQ-5D, BOT-2, CHAQ, gait analysis, and PODCI; or
(b) prior to administration of the sALP:
(i) the patient is identified as having health state IV when the patient has a 6MWT value of less than about 47.8% of a predicted 6MWT value for a healthy subject;
(ii) the patient is identified as having health state III when the patient has a 6MWT value of about 47.8% to about 65.2% of a predicted 6MWT value for a healthy subject;
(iii) the patient is identified as having health state II when the patient has a 6MWT value of about 65.2% to about 82.6% of a predicted 6MWT value for a healthy subject; or
(iv) the patient is identified as having health state I when the patient has a 6MWT value of greater than 82.6% of a predicted 6MWT value for a healthy subject; and
wherein, optionally, after administration of the sALP, the patient has an improved health state.

13-14. (canceled)

15. The method of claim 12, wherein the method further comprises assessing one or more of the following symptoms: elevated blood or urine levels of PPi, PEA, or PLP; osteomalacia, one or more skeletal deformities, hypotonia, muscle weakness, rheumatoid complications, arthritis, pseudogout, waddling gait, ambulatory difficulties, bone pain, pain, premature loss of teeth, hypomineralization, pulmonary hypoplasia, respiratory insufficiency, seizures, hypercalciuria, short stature, and growth delay.

16. The method of claim 2, wherein:
(a) the patient is about 18 years of age or older and the health state of the patient is characterized by performing one or more of the following: the 6MWT, EQ-5D, BOT-2, SF-36, gait analysis, and SF-12; or
(b) prior to administration of the sALP:
(i) the patient is identified as having health state IV when the patient has a 6MWT value of less than about 52.0% of a predicted 6MWT value for a healthy subject;
(ii) the patient is identified as having health state III when the patient has a 6MWT value of about 52.0% to about 68.0% of a predicted 6MWT value for a healthy subject;
(iii) the patient is identified as having health state II when the patient has a 6MWT value of about 68.0% to about 84.0% of a predicted 6MWT value for a healthy subject; or
(iv) the patient is identified as having health state I when the patient has a 6MWT value of greater than 84.0% of a predicted 6MWT value for a healthy subject; and
wherein, optionally, after administration of the sALP, the patient has an improved health state.

17-18. (canceled)

19. The method of any one of claim 16, wherein the method further comprises assessing one or more of the following symptoms: elevated blood or urine levels of PPi, PEA, or PLP, hypomineralization, hypercalciuria, one or more skeletal deformities, hypotonia, muscle weakness, rheumatoid complications, waddling gait, ambulatory difficulties, bone pain, pain, bone fracture, calcium pyrophosphate dihydrate crystal deposition, pseudogout, arthritis, pyrophosphate arthropathy, chondrocalcinosis, calcific periarthritis, and pseudofracture.

20. The method of claim 8, wherein prior to administration of the sALP and after performance of at least the 6MWT:
(i) the patient is identified as having health state IV and the patient has an EQ-5D value of less than about 0.23;
(ii) the patient is identified as having health state III and the patient has an EQ-5D value of about 0.23 to about 0.54;
(iii) the patient is identified as having health state II and the patient has an EQ-5D value of about 0.54 to about 0.67; or
(iv) the patient is identified as having health state I and the patient has an EQ-5D value of greater than about 0.67.

21. (canceled)

22. The method of claim 2, wherein the treatment period is at least three months, at least four months, at least five months, at least six months, at least seven months, at least eight months, at least nine months, at least one year, at least two years, at least three years, at least four years, at least five years, at least six years, at least seven years, at least eight years, at least nine years, or at least ten years, or the lifetime of the patient; particularly at least six months, or at least 96 weeks.

23. (canceled)

24. The method of claim 2, wherein the method further comprises administering the sALP to the patient in a treatment regimen providing about 1 mg/kg/week to about 9 mg/kg/week, preferably 6 mg/kg/week, and/or the patient has an improvement in the health state to at least health state III, II, or I after administration of the sALP.

25. (canceled)

26. The method of claim 2, wherein the sALP is administered;
(a) at an initial dosage of about 2.1 mg/kg/week to about 3.5 mg/kg/week and subsequently is increased to a dosage of about 6 mg/kg/week to about 9 mg/kg/week in the treatment regimen;
(b) one or more times per day, per week, or per month in the treatment regimen;
(c) twice a week, three times a week, four times a week, five times a week, six times a week, or seven times a week in the treatment regimen;
(d) in multiple doses per week, on two days a week, three days a week, four days a week, five days a week, six days a week, or seven days a week;
(e) at a dosage of about 1.3 mg/kg/week, about 2.7 mg/kg/week, or about 6 mg/kg/week in the treatment regimen;
(f) at a dosage of about 2 mg/kg three times a week, about 3 mg/kg two times a week, about 3 mg/kg three times a week, or about 1 mg/kg six times a week in the treatment regimen;
(g) once daily on consecutive or alternating days; and/or
(h) at an initial dosage, wherein the initial dosage is increased after a treatment period of at least six months, at least one year, at least two years, at least three years, or at least four years or longer in the treatment regimen.

27-33. (canceled)

34. The method of claim 2, wherein the method further comprises:
(i) increasing a dose or frequency of administration of the sALP if the patient exhibits a decrease of one or more health state, or does not exhibit an improvement of at least one health state, in the health state after treatment with the sALP;
(ii) maintaining a dose or frequency of administration of the sALP if the patient exhibits the same health state or an improvement of at least one health state after treatment with the sALP; or
(iii) reducing a dose or frequency of administration of the sALP if the patient exhibits an improvement of more than one health state after treatment with the sALP.

35. The method of claim 2, wherein the sALP:
(a) comprises or consists of the amino acid sequence of SEQ ID NO:
(b) is administered in a composition comprising at least one pharmaceutically acceptable carrier, diluent, or excipient;
(c) is physiologically active toward PEA, PPi, and PLP;
(d) is catalytically competent to improve skeletal mineralization in bone;
(e) is the soluble extracellular domain of an alkaline phosphatase; and/or
(f) is administered subcutaneously, intramuscularly, intravenously, orally, nasally, sublingually, intrathecally, or intradermally.

36-37. (canceled)

38. The method of claim 35, wherein the at least one pharmaceutically acceptable carrier, diluent, or excipient is saline or comprises sodium chloride and sodium phosphate, wherein, optionally, the at least one pharmaceutically acceptable carrier, diluent, or excipient comprises about 150 mM sodium chloride and about 25 mM sodium phosphate.

39-46. (canceled)

47. The method of claim 2, wherein the patient is an asfotase alfa treatment naïve patient and/or wherein the patient is a human.

48. (canceled)

49. The method of claim 1, wherein the health state is I, II, III, or IV.

50. The method of claim 49, wherein the method further comprises:
(c) administering a treatment regimen comprising at least 0.5 mg/kg/week of a soluble alkaline phosphatase (sALP) to the patient for a treatment period of at three months, wherein the sALP comprises an amino acid sequence having at least 95% sequence identity to the amino acid sequence of SEQ ID NO: 1.

51-214. (canceled)


SEQUENCE LISTING (20 sequences)

SEQ ID NO: 1 - protein, 726 aa, Artificial Sequence
  Synthetic Construct
LVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSLKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKDIDDDDDDDDDD

SEQ ID NO: 2 - protein, 524 aa, Homo sapiens
MISPFLVLAIGTCLTNSLVPEKEKDPKYWRDQAQETLKYALELQKLNTNVAKNVIMFLGDGMGVSTVTAARILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLMHNIRDIDVIMGGGRKYMYPKNKTDVEYESDEKARGTRLDGLDLVDTWKSFKPRYKHSHFIWNRTELLTLDPHNVDYLLGLFEPGDMQYELNRNNVTDPSLSEMVVVAIQILRKNPKGFFLLVEGGRIDHGHHEGKAKQALHEAVEMDRAIGQAGSLTSSEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYVPHVMAYAACIGANLGHCAPASSAGSLAAGPLLLALALYPLSVLF

SEQ ID NO: 3 - protein, 524 aa, Homo sapiens [sequence omitted]

SEQ ID NO: 4 - protein, 524 aa, Homo sapiens; identical to SEQ ID NO: 2

SEQ ID NO: 5 - protein, 524 aa, Homo sapiens; identical to SEQ ID NO: 2

SEQ ID NO: 6 - protein, 524 aa, Homo sapiens [sequence omitted]

SEQ ID NO: 7 - protein, 652 aa, Macaca mulatta
MPTVKTKQESHAGSGSGPRLAERKGRVGAARRQSPRAPGGGLPGPRSGPAAAFIRRRGRWPGPRCAPATPRPRSRLCAPTRLCLDEPSSVLCAGLEHQLTSDHCQPTPSHPRRSHLWASGIKQVLGCTMISPFLVLAIGTCLTNSLVPEKEKDPKYWRDQAQETLKYALELQKLNTNVAKNVIMFLGDGMGVSTVTATRILKGQLHHNPGEETRLEMDKFPFVALSKTYNTNAQVPDSAGTATAYLCGVKANEGTVGVSAATERSRCNTTQGNEVTSILRWAKDAGKSVGIVTTTRVNHATPSAAYAHSADRDWYSDNEMPPEALSQGCKDIAYQLVHNIRDIDVIMGGGRKYMYPKNKTDVEYEIDEKARGTRLDGLDLVNIWKSFKPRHKHSHFIWNRTELLTLDPHNVDYLLGLFEPGDMEYELNRNNVTDPSLSEMVVVAIQILRKNPKGFFLLVEGGRIDHGHHEGKAKQALHEAVEMDRAIGQAGSMTSLEDTLTVVTADHSHVFTFGGYTPRGNSIFGLAPMLSDTDKKPFTAILYGNGPGYKVVGGERENVSMVDYAHNNYQAQSAVPLRHETHGGEDVAVFSKGPMAHLLHGVHEQNYIPHVMAYAACIGANLDHCAPASSAGSLAAGPLLLPLALFPLSILF

SEQ ID NO: 8 - protein, 524 aa, Rattus norvegicus
MILPFLVLAIGPCLTNSFVPEKEKDPSYWRQQAQETLKNALKLQKLNTNVAKNIIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMRPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNPKGFFLLVEGGRIDHGHHEGKAKQALHEAVEMDEAIGKAGTMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWASSASSPSPGALLLPLALFPLRTLF

SEQ ID NO: 9 - protein, 502 aa, Canis lupus familiaris
EKDPKYWRDQAQQTLKYALRLQNLNTNVAKNVIMFLGDGMGVSTVTATRILKGQLHHNPGEETRLEMDKFPYVALSKTYNTNAQVPDSAGTATAYLCGVKANEGTVGVSAATQRTHCNTTQGNEVTSILRWAKDAGKSVGIVTTTRVNHATPSAAYAHSADRDWYSDNEMPPEALSQGCKDIAYQLMHNVKDIEVIMGGGRKYMFPKNRTDVEYEMDEKSTGARLDGLNLIDIWKNFKPRHKHSHYVWNRTELLALDPYTVDYLLGLFDPGDMQYELNRNNVTDPSLSEMVEIAIKILSKKPRGFFLLVEGGRIDHGHHEGKAKQALHEAVEMDRAIGKAGVMTSLEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANQDHCASASSAGGPSPGPLLLLLALLPVGILF

SEQ ID NO: 10 - protein, 252 aa, Sus scrofa
AELLALDPHTVDYLLGLFEPGDMQYELNRNNVTDPSLSEMVEMAIRILIKNPKGFFLLVEGGRIDHGHHEGKAKQALHEAVEMDRAIEQAGSMTSVEDTLTVVTADHSHVFTFGGYTPRGNSIFGLAPMVSDTDKKPFTAILYGNGPGYKVVGGERENVSMVDYAHDNYQAQSAVPLRHETHGGEDVAIFARGPMAHLLHGVHEQNYIPHVMAYAACVGANRDHCASASSSGSPSPGPLLLLLALLPLGILF

SEQ ID NO: 11 - protein, 524 aa, Mus musculus
MISPFLVLAIGTCLTNSFVPEKERDPSYWRQQAQETLKNALKLQKLNTNVAKNVIMFLGDGMGVSTVTAARILKGQLHHNTGEETRLEMDKFPFVALSKTYNTNAQVPDSAGTATAYLCGVKANEGTVGVSAATERTRCNTTQGNEVTSILRWAKDAGKSVGIVTTTRVNHATPSAAYAHSADRDWYSDNEMPPEALSQGCKDIAYQLMHNIKDIDVIMGGGRKYMYPKNRTDVEYELDEKARGTRLDGLDLISIWKSFKPRHKHSHYVWNRTELLALDPSRVDYLLGLFEPGDMQYELNRNNLTDPSLSEMVEVALRILTKNLKGFFLLVEGGRIDHGHHEGKAKQALHEAVEMDQAIGKAGAMTSQKDTLTVVTADHSHVFTFGGYTPRGNSIFGLAPMVSDTDKKPFTAILYGNGPGYKVVDGERENVSMVDYAHNNYQAQSAVPLRHETHGGEDVAVFAKGPMAHLLHGVHEQNYIPHVMAYASCIGANLDHCAWAGSGSAPSPGALLLPLAVLSLRTLF

SEQ ID NO: 12 - protein, 524 aa, Bos taurus
MISPFLLLAIGTCFASSLVPEKEKDPKYWRDQAQQTLKNALRLQTLNTNVAKNVIMFLGDGMGVSTVTAARILKGQLHHSPGEETKLEMDKFPYVALSKTYNTNAQVPDSAGTATAYLCGVKANEGTVGVSAATQRSQCNTTQGNEVTSILRWAKDAGKSVGIVTTTRVNHATPSASYAHSADRDWYSDNEMPPEALSQGCKDIAYQLMHNIKDIEVIMGGGRKYMFPKNRTDVEYELDEKARGTRLDGLNLIDIWKSFKPKHKHSHYVWNRTDLLALDPHSVDYLLGLFEPGDMQYELNRNNATDPSLSEMVEMAIRILNKNPKGFFLLVEGGRIDHGHHEGKAKQALHEAVEMDQAIGQAGAMTSVEDTLTVVTADHSHVFTFGGYTPRGNSIFGLAPMVSDTDKKPFTAILYGNGPGYKVVGGERENVSMVDYAHNNYQAQSAVPLRHETHGGEDVAVFAKGPMAHLLHGVHEQNYIPHVMAYAACIGANRDHCASASSSGSPSPGPLLLLLALLPLGSLF

SEQ ID NO: 13 - protein, 524 aa, Bos taurus; identical to SEQ ID NO: 12

SEQ ID NO: 14 - protein, 124 aa, Bos taurus
DPKYWRDQAQQTLKNALGLQKLNTKVAKNVILFLGDGMGVSTVTAARILKGQLHHNPGEETRLEMDKFPFVALSKTYNTNAQVPDSAGTAPHPVRVKAMRAPWGEPHQRQCNTRRATSTHLLAG

SEQ ID NO: 15 - protein, 524 aa, Felis catus
MISPFLVLAIGTCLTNSLVPEKEKDPKYWRDQAQQTLKNALRLQKLNTNVVKNVIMFLGDGMGVSTVTAARILKGQLHHNPGEETRLEMDKFPYVALSKTYNTNAQVPDSAGTATAYLCGVKANEGTVGVSAATQRTQCNTTQGNEVTSILRWAKDSGKSVGIVTTTRVNHATPSAAYAHSADRDWYSDNEMPPEALSQGCKDIAYQLMHNVRDIEVIMGGGRKYMFPKNRTDVEYEMDEKARGTRLDGLNLVDIWKSFKPRHKHSHYVWNRTELLTLDPYGVDYLLGLFEPGDMQYELNRNSTTDPSLSEMVEIAIKILSKNPKGFFLLVEGGRIDHGHHEGKAKQALHEAVEMDQAIGRAGAMTSVEDTLTIVTADHSHVFTFGGYTPRGNSIFGLAPMVSDTDKKPFTSILYGNGPGYKVVGGERENVSMVDYAHNNYQAQSAVPLRHETHGGEDVAVFAKGPMAHLLHGVHEQNYIPHVMAYAACIGANLDHCASASSAGGPSPGPLFLLLALPSLGILF

SEQ ID NO: 16 - protein, 532 aa, Homo sapiens
MQGPWVLLLLGLRLQLSLGIIPVEEENPDFWNRQAAEALGAAKKLQPAQTAAKNLIIFLGDGMGVSTVTAARILKGQKKDKLGPETFLAMDRFPYVALSKTYSVDKHVPDSGATATAYLCGVKGNFQTIGLSAAARFNQCNTTRGNEVISVMNRAKKAGKSVGVVTTTRVQHASPAGAYAHTVNRNWYSDADVPASARQEGCQDIATQLISNMDIDVILGGGRKYMFPMGTPDPEYPDDYSQGGTRLDGKNLVQEWLAKHQGARYVWNRTELLQASLDPSVTHLMGLFEPGDMKYEIHRDSTLDPSLMEMTEAALLLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDGETHAGEDVAVFARGPQAHLVHGVQEQTFIAHVMAFAACLEPYTACDLAPRAGTTDAAHPGPSVVPALLPLLAGTLLLLGTATAP

SEQ ID NO: 17 - protein, 535 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGRSVVPALLPLLAGTLLLLETATAP

SEQ ID NO: 18 - protein, 532 aa, Homo sapiens; identical to SEQ ID NO: 16

SEQ ID NO: 19 - protein, 528 aa, Homo sapiens
MQGPWVLLLLGLRLQLSLGVIPAEEENPAFWNRQAAEALDAAKKLQPIQKVAKNLILFLGDGLGVPTVTATRILKGQKNGKLGPETPLAMDRFPYLALSKTYNVDRQVPDSAATATAYLCGVKANFQTIGLSAAARFNQCNTTRGNEVISVMNRAKQAGKSVGVVTTTRVQHASPAGTYAHTVNRNWYSDADMPASARQEGCQDIATQLISNMDIDVILGGGRKYMFPMGTPDPEYPADASQNGIRLDGKNLVQEWLAKHQGAWYVWNRTELMQASLDQSVTHLMGLFEPGDTKYEIHRDPTLDPSLMEMTEAALRLLSRNPRGFYLFVEGGRIDHGHHEGVAYQALTEAVMFDDAIERAGQLTSEEDTLTLVTADHSHVFSFGGYTLRGSSIFGLAPSKAQDSKAYTSILYGNGPGYVFNSGVRPDVNESESGSPDYQQQAAVPLSSETHGGEDVAVFARGPQAHLVHGVQEQSFVAHVMAFAACLEPYTACDLAPPACTTDAAHPVAASLPLLAGTLLLLGASAAP

SEQ ID NO: 20 - protein, 227 aa, Artificial Sequence
  Synthetic Construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK